(19) United States
(12) Patent Application Publication
(10) Pub. No.: US 2016/0022244 A1
(43) Pub. Date: Jan. 28, 2016

(54) MEDICAL PROBES HAVING INTERNAL HYDROPHILIC SURFACES

(71) Applicants: COLIBRI TECHNOLOGIES INC., Toronto (CA); SUNNYBROOK RESEARCH INSTITUTE, Toronto (CA)

(72) Inventors: Brian Courtney, Toronto (CA); Amandeep Thind, Toronto (CA); Alan Soong, Etobicoke (CA); Sneha Mathrani, Markham (CA)

(21) Appl. No.: 14/776,957

(22) PCT Filed: Mar. 14, 2014
(86) PCT No.: PCT/CA2014/050248

§ 371 Date: Sep. 15, 2015

Related U.S. Application Data
(60) Provisional application No. 61/801,053, filed on Mar. 15, 2013.

(51) Int. Cl. A61B 8/12; A61B 8/08; A61M 25/00; A61B 8/00

(52) U.S. Cl. CPC A61B 8/12; A61B 8/4416; A61B 8/0883; A61B 8/4281; A61B 8/445; A61B 8/4483; A61M 25/0045; A61M 2025/0046; A61M 2025/0047

(57) ABSTRACT

Medical probes having an inner fluidic path for flowing an internal liquid therein are disclosed, in which at least one internal surface in flow communication with the inner fluidic path is hydrophilic for the reduction of bubble adhesion thereto. In some embodiments, imaging probes are described, in which an internal surface in flow communication with an internal fluidic path, and through which imaging energy propagates, is coated with a hydrophilic layer that has a thickness and/or an acoustic impedance for reducing an impedance mismatch. Various configurations are described, including embodiments in which hydrophobic bubble trapping surface regions are included in addition to the hydrophilic surface regions. In some embodiments, a medical probe may have an inner lumen defined by an inner fluidic conduit, where at least a portion of the inner surface of the inner fluidic conduit is hydrophilic.

### CROSS-REFERENCE TO RELATED APPLICATION

This application claims priority to U.S. Provisional Application No. 61/801,053, titled “SURFACE POLARIZATION OF INNER SURFACES OF IMAGING AND THERAPEUTIC ULTRASOUND PROBES” and filed on Mar. 15, 2013, the entire contents of which is incorporated herein by reference.

### BACKGROUND

The present disclosure relates to medical probes, and more particularly, the present disclosure relates to medical probes, such as catheters, in which a fluid is transported within a portion of the probe.

Medical probes, such as catheters, are commonly used in minimally-invasive procedures for the diagnosis and treatment of medical conditions. Such procedures may involve the use of intraluminal, intracavity, intravascular, and intracardiac catheters and related systems. When performing such procedures, imaging and treatment catheters are often inserted percutaneously into the body and into an accessible vessel of the vascular system at a site remote from the vessel or organ to be diagnosed and/or treated. The catheter is then advanced through the vessels of the vascular system to the region of the body to be treated.

The catheter may be further equipped with an imaging device employing an optical imaging modality, such as optical coherence tomography.

For example, an ultrasound imaging device may be employed to locate and diagnose a diseased portion of the body, such as a stenosed region of an artery. The catheter may also be provided with a therapeutic device, such as those used for performing interventional techniques including balloon angioplasty, laser ablation, rotational atherectomy, directional atherectomy, acoustic ablation, and the like.

### SUMMARY

Medical probes having an inner fluidic path for flowing an internal liquid therein are disclosed, in which at least one internal surface in flow communication with the inner fluidic path is hydrophilic for the reduction of bubble adhesion thereto. In some embodiments, imaging probes are described, in which an internal surface in flow communication with an internal fluidic path, and through which imaging energy propagates, is coated with a hydrophilic layer that has a thickness and/or an acoustic impedance for reducing an impedance mismatch. Various configurations are described, including embodiments in which hydrophobic bubble trapping surface regions are included in addition to the hydrophilic surface regions. In some embodiments, a medical probe may have an inner lumen defined by an inner fluidic conduit, where at least a portion of the inner surface of the inner fluidic conduit is hydrophilic.

An imaging probe comprising:

a hollow sheath;

an imaging assembly housed within said hollow sheath, wherein said imaging assembly is positionable remote from a proximal region of said hollow sheath, and wherein said imaging assembly is configured to emit and/or receive imaging energy;

at least one fluidic path provided within said hollow sheath, wherein said fluidic path extends longitudinally within said hollow sheath from said proximal region and is in flow communication with said imaging assembly;

at least one port associated with said hollow sheath for introducing a liquid into said fluidic path;

wherein at least one imaging surface, through which imaging energy is transmitted, and which is in flow communication with said fluidic path, comprises a hydrophilic layer.

In another aspect, there is provided an imaging probe comprising:

a hollow sheath;

an ultrasonic transducer housed within said hollow sheath, wherein said ultrasonic transducer is positionable remote from a proximal region of said hollow sheath, and wherein said ultrasonic transducer is configured to emit and/or receive imaging energy;

at least one fluidic path provided within said hollow sheath, wherein said fluidic path extends longitudinally within said hollow sheath from said proximal region and is in flow communication with said ultrasonic transducer;

at least one port associated with said hollow sheath for introducing a liquid into said fluidic path;

wherein an emitting surface of said ultrasonic transducer is configured to be hydrophilic.

In another aspect, there is provided a medical probe comprising:

a hollow sheath;

an ultrasonic transducer housed within said hollow sheath, wherein said ultrasonic transducer is positionable remote from a proximal region of said hollow sheath, wherein said ultrasonic transducer is configured to emit ultrasonic energy into an external region;

at least one fluidic path provided within said hollow sheath, wherein said fluidic path extends longitudinally within said hollow sheath from said proximal region and is in flow communication with said ultrasonic transducer;

at least one port associated with said hollow sheath for introducing a liquid into said fluidic path;

wherein at least one internal surface that is in flow communication with said fluidic path, and through which the ultrasonic energy propagates from said ultrasonic transducer, comprises a hydrophilic layer configured to reduce an impedance mismatch for ultrasonic energy propagating through said internal surface when said fluidic path is filled with a liquid.

In another aspect, there is provided a medical probe comprising:

a hollow sheath;

a functional device housed within said hollow sheath, wherein said functional device is rotatable and positionable remote from a proximal region of said hollow sheath;

at least one fluidic path provided within said hollow sheath, wherein said fluidic path extends longitudinally within said hollow sheath from said proximal region and is in flow communication with said functional device;

at least one port associated with said hollow sheath for introducing a liquid into said fluidic path;

wherein one or more stationary components having a stationary internal surface in fluid communication with said fluidic path are configured such that at least a portion of said stationary internal surface is hydrophilic; and

wherein one or more rotatable components having a rotatable internal surface in fluid communication with said fluidic path are configured such that at least a portion of said rotatable internal surface is hydrophilic.

In another aspect, there is provided a medical probe comprising:

a hollow sheath;

a functional device housed within said hollow sheath, wherein said functional device is positionable remote from a proximal region of said hollow sheath;
at least one fluidic path provided within said hollow sheath, wherein said fluidic path extends longitudinally within said hollow sheath from said proximal region and is in flow communication with said functional device;
at least one port associated with said hollow sheath, wherein said port is in flow communication with said fluidic path;

wherein at least one internal surface defining said fluidic path comprises:
at least one hydrophilic surface region for reducing adhesion of bubbles that could impair the operation of said functional device, wherein at least a portion of said at least one hydrophilic surface region is provided near said functional device when said functional device is employed during a medical procedure; and
at least one hydrophobic surface region for trapping bubbles and preventing bubbles from interfering with the operation of said functional device, wherein said at least one hydrophobic surface region is provided at a location between said functional device and said port.

In another aspect, there is provided a medical probe comprising:

a hollow sheath;

a functional device housed within said hollow sheath, wherein said functional device is positionable remote from the proximal end of said hollow sheath;

an inner fluidic conduit housed within said hollow sheath;

a first port in flow communication with a lumen of said inner fluidic conduit;

a second port in flow communication with a lumen of said hollow sheath; and

a fluidic path defined by:
an outer lumen formed between an inner surface of said hollow sheath and an outer surface of said inner fluidic conduit; and
an inner lumen formed within said inner fluidic conduit;

wherein said inner lumen is in fluid communication with said outer lumen near a region remote from the proximal end; and

wherein at least a portion of an inner surface of said inner fluidic conduit is hydrophilic.

In another aspect, there is provided a medical probe comprising:

a hollow sheath;

a functional device housed within said hollow sheath, wherein said functional device is rotatable and positionable remote from a proximal region of said hollow sheath;

at least one fluidic path provided within said hollow sheath, wherein said fluidic path extends longitudinally within said hollow sheath from said proximal region and is in flow communication with said functional device;

at least one port associated with said hollow sheath for introducing a liquid into said fluidic path;

wherein one or more rotatable components having a rotatable internal surface in fluid communication with said fluidic path are configured such that at least a portion of said rotatable internal surface is hydrophilic.

In another aspect, there is provided a medical probe comprising:

a hollow sheath;

a functional assembly housed within said hollow sheath, wherein said functional assembly is positionable remote from a proximal region of said hollow sheath, and wherein said functional assembly is configured to emit and/or receive energy;

at least one fluidic path provided within said hollow sheath, wherein said fluidic path extends longitudinally within said hollow sheath from said proximal region and is in flow communication with said functional assembly;

at least one port associated with said hollow sheath for introducing a liquid into said fluidic path;

wherein at least one surface that is in flow communication with said fluidic path comprises a hydrophilic layer configured to reduce an impedance mismatch for energy propagating therethrough when said fluidic path is filled with a liquid.

In another aspect, there is provided an imaging probe comprising:

a hollow sheath;

an imaging assembly housed within said hollow sheath, wherein said imaging assembly is positionable remote from a proximal region of said hollow sheath, and wherein said imaging assembly is configured to emit and/or receive imaging energy;

an imaging region containing said imaging assembly, wherein said imaging region can be filled with liquid;

wherein at least one surface within said imaging region comprises a hydrophilic layer configured to reduce an impedance mismatch for imaging energy propagating therethrough when said imaging region is filled with a liquid.

In another aspect, there is provided a medical probe comprising:

a hollow sheath;

a functional device housed within said hollow sheath, wherein said functional device is positionable remote from a proximal region of said hollow sheath;

at least one fluidic path provided within said hollow sheath, wherein said fluidic path extends longitudinally within said hollow sheath from said proximal region and is in flow communication with said functional device;

at least one port associated with said hollow sheath, wherein said port is in flow communication with said fluidic path;

wherein an internal surface of said hollow sheath comprises at least one hydrophilic surface region for reducing adhesion of bubbles that could impair the operation of said functional device, wherein at least a portion of said at least one hydrophilic surface region is provided near a location where said functional device is positioned during a medical procedure.

A further understanding of the functional and advantageous aspects of the disclosure can be realized by reference to the following detailed description and drawings.

### BRIEF DESCRIPTION OF THE DRAWINGS

Embodiments of the disclosure will now be described, by way of example only, with reference to the drawings, in which:

FIG. 1 is a schematic of an imaging system including ultrasound and optical components.

FIG. 2 is a perspective drawing of a flexible imaging probe with an adapter, conduit, and imaging assembly.

FIG. 2a is a cross sectional view of the mid-section of the imaging probe of FIG. 2 taken along the dotted line.

FIG. 2b is a magnified and expanded drawing of the distal region of the imaging probe of FIG. 2.

FIGS. 3a-3d describe embodiments of techniques for causing tilting of a tiltable member.

FIG. 3a shows a longitudinal cutaway of a catheter in which the tilting is caused by centripetal motion.

FIG. 3b shows a cross-sectional cutaway of the catheter shown in FIG. 3a.

FIG. 3c shows the catheter of FIG. 3a and the resulting tilting caused by rotating the scanning assembly at a faster rate than that of FIG. 3a.

FIG. 3d shows a cross-sectional cutaway of the catheter shown in FIG. 3c.

FIG. 3e shows a longitudinal cutaway of a catheter in which the tilting is controlled using one or more magnets.

FIG. 3f shows a cross-sectional cutaway of the catheter in FIG. 3e.

FIG. 3g shows the catheter of FIG. 3e and the resulting deflection caused by magnetism.

FIG. 3h shows a cross-sectional cutaway of the catheter in FIG. 3g.

FIG. 3i shows a potential scanning pattern for generating 3D images with imaging angle information.

FIG. 3j illustrates a control system in which the angle sensing transducer is employed to provide feedback for controlling a direction of the emitted imaging beam.

FIG. 3k shows an implementation of a system using a torsional spring as a restoring mechanism.

FIG. 4a shows the unmodified inner surfaces of a distal dome, catheter sheath, inner conduit and proximal flush port with the presence of air bubbles adhering to the surfaces thereof.

FIG. 4b shows an inner region of the hollow shaft configured to be hydrophilic by adding a hydrophilic layer.

FIG. 4c shows an inner region of the hollow shaft configured to be hydrophilic by impregnating shaft material with hydrophilic additives.

FIG. 5a shows the fluid path, relevant catheter components of an ultrasound imaging probe, distal dome, catheter sheath, inner conduit, torque cable and proximal flush port having not been induced to have hydrophilic properties, with the presence of air bubbles adhering to the surfaces thereof.

FIG. 5b shows the distal dome of the catheter having a hydrophilic inner surface free of air bubbles in the region through which ultrasound energy could propagate during operation.

FIG. 5c shows the inner surfaces of the distal dome, full length of catheter sheath, and full length of the inner conduit treated to be hydrophilic and free of adhering bubbles.

FIG. 5d shows the inner surfaces of the distal dome, full length of catheter sheath, full length of the inner conduit, and torque cable, treated to be hydrophilic and free of adhering bubbles.

FIG. 5e shows the inner surfaces of the distal dome, full length of catheter sheath, full length of the inner conduit, full length of the torque cable, and proximal flush port treated to be hydrophilic and free of adhering bubbles.

FIG. 6a shows the inner surfaces of the distal dome and partial lengths of the catheter sheath and inner conduit treated to be hydrophilic and free of adhering bubbles, and other partial lengths of catheter sheath and inner conduit at the proximal region treated to be hydrophobic with adhered bubbles.

FIG. 6b shows partial lengths of the inner surfaces of the hollow shaft and inner conduit at the proximal region configured to be hydrophobic by adding a hydrophobic layer.

FIG. 6c shows partial lengths of the inner surfaces of the hollow shaft and inner conduit at the proximal region configured to be hydrophobic by increasing the surface roughness.

FIG. 7 shows two regions of the inner surfaces of the catheter sheath and inner conduit configured to be hydrophilic and two regions of each of the inner surfaces of the catheter sheath and inner conduit away from the distal region, configured to be hydrophobic.

FIG. 8 shows a hydrophobic trapping region created on the catheter sheath and inner conduit, in close proximity of the functional device, at a location remote from the proximal region.

FIG. 9 shows an inner surface of a catheter sheath region located away from the distal region through which transmission of ultrasound energy occurs, known as the imaging region, rendered to be hydrophilic.

FIG. 10a shows the transducer's emitting surface impeded by an air bubble adherent to the inner surface of distal dome in the absence of a hydrophilic surface.

FIG. 10b shows the transducer's emitting surface free of obstructions and mobile, free-floating bubbles that can be more easily flushed in the presence of a hydrophilic inner surface.

FIG. 11 shows a hydrophilic layer applied to the distal dome, inner and outer surfaces of a housing that can house an imaging assembly.

FIG. 12 shows a hydrophilic coating applied to the inner surfaces of the distal dome and an imaging assembly housing, and a hydrophobic coating applied to at least a portion of the outer surface.

FIG. 13 shows a hydrophilic coating affixed to every outer surface of the transducer and a spring that provides a restoring force mechanism for enabling variable tilt angles for 3D imaging, with the distal dome also rendered to be hydrophilic.

FIG. 14 shows a hydrophilic surface imparted onto the inner surface of the distal dome and a hydrophilic layer applied to the transducer and reflective surface of the imaging assembly designed to facilitate an estimation of the transducer's tilt angle.

FIG. 15 shows a side-viewing ultrasound imaging transducer where the emitting surface of the ultrasound transducer is selectively coated with a hydrophilic coating, with the distal dome also rendered to be hydrophilic.

FIG. 16 shows the inner surface of the distal dome of a catheter modified to be hydrophilic, in which magnetic drive mechanism provides variable tilt to an ultrasound transducer.

FIG. 17 shows an acoustically and optically compatible hydrophilic modification of the distal dome of a catheter which combines ultrasound and optical imaging for its imaging capabilities.

FIG. 18 shows relevant catheter components of an optical imaging probe with a hydrophilic layer applied to the optical prism, optical reflector, and also imaging assembly housing, with the distal dome of the catheter modified to be hydrophilic.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately”, when used in conjunction with ranges of dimensions of particles, compositions of mixtures or other physical properties or characteristics, are meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present disclosure.

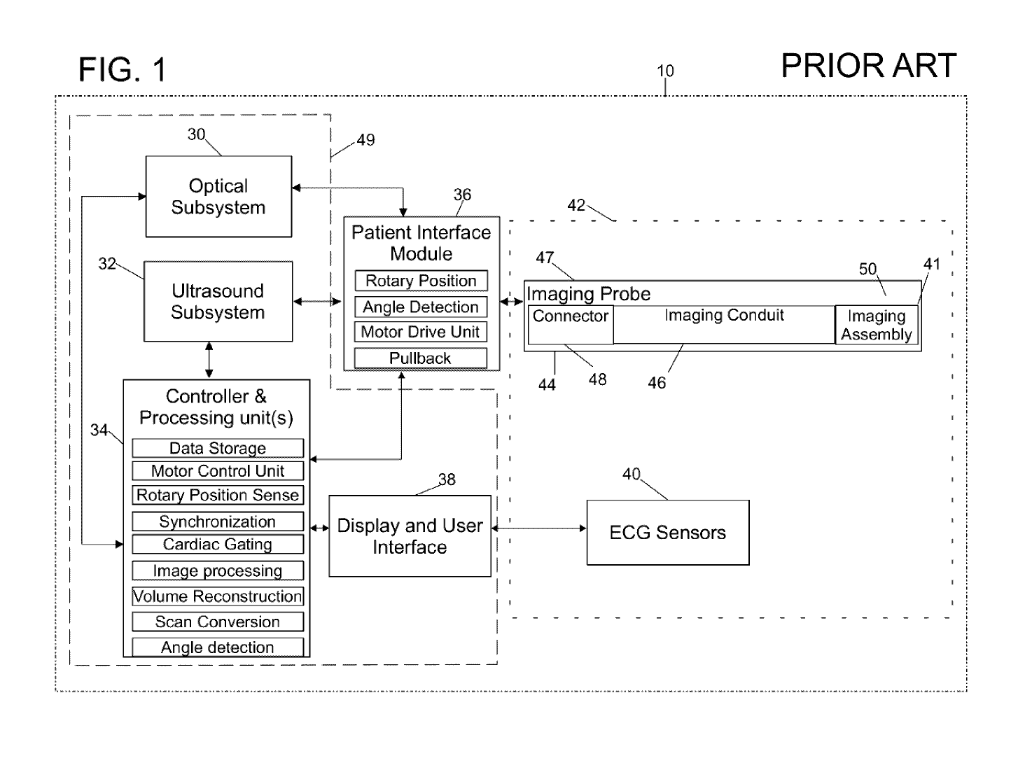

A brief review of minimally invasive imaging systems is provided with reference to FIGS. 1 to 3, by way of example. Referring first to FIG. 1, an imaging system is shown at 10 comprising imaging probe 44, which connects via patient interface module 36 to image processing and display system 49. Image processing and display system 49 includes hardware to support one or more imaging modalities, such as ultrasound, optical coherence tomography, angioscopy, infrared imaging, near infrared imaging, Raman spectroscopy-based imaging, or fluorescence imaging. Specific embodiments of ultrasonic imaging probes and combined ultrasonic and optical imaging probes are disclosed by Courtney et al. in US Patent Publication No. 20080177183, titled “Imaging Probe with Combined Ultrasound and Optical Means of Imaging” and filed on Jan. 22, 2008, US Patent Publication No. 20080177138, titled “Scanning Mechanisms for Imaging Probe” and filed on Jan. 22, 2008 and US Patent Publication No. 20090264768, titled “Scanning Mechanisms for Imaging Probe” and filed on Mar. 27, 2009, each of which are incorporated herein by reference in their entirety.

Controller and processing unit 34 is employed to facilitate the coordinated activity of the many functional units of the system, and may contain some or all of the components shown in the Figure and listed herein. An operator interacts with system 50 via display and/or user interface 38. System 10 may further include electrode sensors 40 to acquire electrocardiogram signals from the body of the patient being imaged.

Optical subsystem 30, if included in a particular implementation of an imaging system, may include any or all of the following components: interferometer components, one or more optical reference arms, optical multiplexors, optical demultiplexers, light sources, photodetectors, spectrometers, polarization filters, polarization controllers, timing circuitry, analog to digital converters, parallel processing arrays and other components known to facilitate any of the optical imaging techniques. Ultrasound subsystem 32 may include any or all of the following components: pulse generators, electronic filters, analog to digital converters, parallel processing arrays, envelope detectors, amplifiers including time gain compensation amplifiers and other components known to facilitate acoustic imaging techniques.

It is to be understood that patient interface module 36 and controller and processing units 34 are but one example illustration of the selection and organization of hardware subsystems, and that many other implementations are possible. For example, patient interface module 36 may be housed with controller and processing units 34 within processing and display system 49.

Example imaging probe 44 includes an imaging assembly 50, optional imaging conduit 46 along a substantial portion of its length, and connector 48 at its proximal region 47. Imaging assembly 50 is located at a location remote from the proximal region, for example, near distal end 41 of imaging probe 44. Imaging assembly 50 generally refers to the components of the imaging probe 44 from which the signals (either acoustic, optical or both) are collected for the purposes of imaging a region that is proximate to imaging assembly 50. Imaging assembly 50 may house transducers for transmitting and/or receiving imaging radiation. The emitter and receiver may be a single component, as is often the case with a piezoelectric transducer.

In the case of optical imaging, imaging assembly 50 typically contains the distal tip of a fiber optic, as well as a combination of optical components such as a lens (for instance, a ball lens or a GRIN lens). A mirror and/or prism may be included for use in beam delivery and/or collection. Optionally, there may be an optical detector, such as a CCD array, or an optical light source, such as one or more LEDs, incorporated directly in the imaging assembly that may obviate the need for one or more fiber optics in an optical imaging probe.

Imaging probe 44 may contain ports at one or more points along its length to facilitate flushing. Moreover, imaging assembly 50, connector 48 and/or imaging conduit 46 may be filled and/or surrounded with a fluid such as saline, and may be flushed.

Imaging conduit 46 includes at least one conductive wire (optionally two or more) that connect an emitter and/or receiver via connection to an adapter, herein referred to as patient interface module 36.

Patient interface module 36 facilitates transmission of signals within any fibers and/or wires to the appropriate image processing units. It may contain a motor drive unit for imparting rotational motion to the components of the imaging mechanism.

In many applications, it can be important to optimize the geometry of a minimally invasive probe so that it is as small as reasonably possible to achieve its desired purpose. Current IVUS and ICE probes are approximately 0.9 to 4 mm in diameter and the smaller sizes of probes can be delivered more distally within the vascular tree of the coronary anatomy as the vessel caliber tapers down or as diseased vessels are stenosed. Furthermore, within the cardiac anatomy, smaller probes (such as those with a diameter less than about 3.4 mm) can be readily advanced across the atrial septum into the left atrium of the heart. Thus, smaller sizes generally allow for delivery of the device into a larger portion of the coronary or cardiac anatomy. It is therefore desirable for a probe and its components to be contained within a minimal outer diameter to enable imaging, such as using imaging performed with the scanning mechanisms described by Courtney et al. (US Issued U.S. Pat. No. 8,214,010, which is incorporated herein by reference in its entirety).

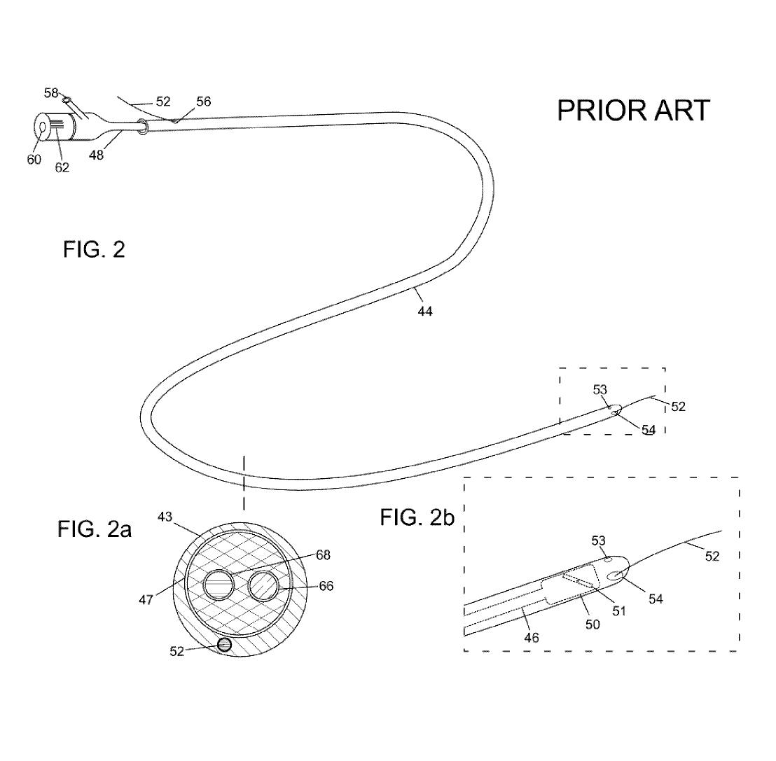

FIG. 2 is a perspective drawing of a flexible catheter containing fiber optic 66 and co-axial electrical cable 68. The proximal connector contains fiber optic connection joint 60 that can be received by patient interface module 36 to optically couple imaging fiber optic 66 to image processing and display system 49. Electrical connectors 62 allow one or more electrical conduits to be connected to the ultrasound circuitry and/or controller and processing units. In applications in which the imaging conduit rotates around its longitudinal axis, there may be a need to couple the rotating components of the imaging fiber optic with a relatively stationary fiber optic that connects to image processing and display system 49. This coupling can be achieved with the use of a fiber optic rotary joint incorporated either as part of the proximal connector of imaging probe 48 or as part of patient interface module 36. Similarly, there may need to be a mechanism for coupling the rotating components of the electrical system with relatively stationary electrical components that connect to image processing and display system 49. This can be achieved through the use of one or more electrical slip rings or slip ring channels.

FIG. 2a shows a cross sectional view of the middle section of the catheter shown in FIG. 2 taken along the dotted vertical line. The cross section shows the optional fiber optic 66, optional guidewire 52, imaging conduit lumen 47, external sheath 43, which is a hollow, flexible elongate shaft made of physiologically compatible material and having a diameter suitable to permit insertion of the hollow elongate shaft into bodily lumens and cavities, and co-axial wiring 68. The expanded detailed view of the distal region of the imaging probe 44 in FIG. 2b shows the imaging assembly 50 which optionally includes a tiltable member 51, distal end of the optional guidewire 52 extended beyond the end of the external sheath 43 and a flush port 53 near the end of the sheath 43. In FIG. 2, the proximal region of the imaging probe 44 includes an optional guidewire port 56 into which the guidewire 52 is inserted and the connector assembly 48 includes a flush port 58 and electrical contacts 62 along with the connector body. An optional guidewire port 54 is seen in FIG. 2b.

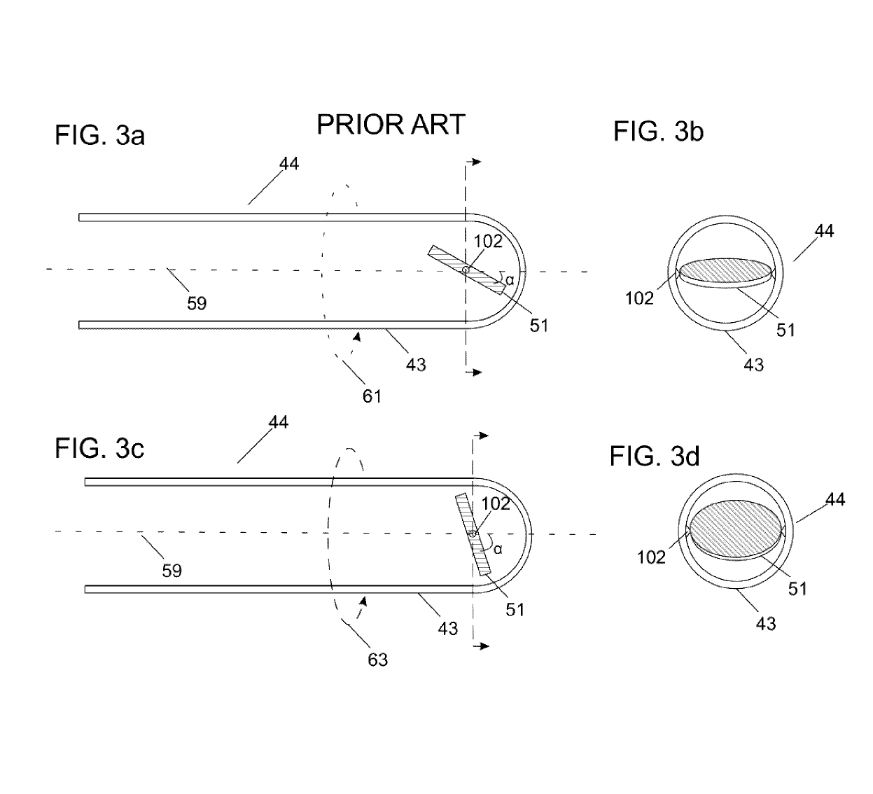

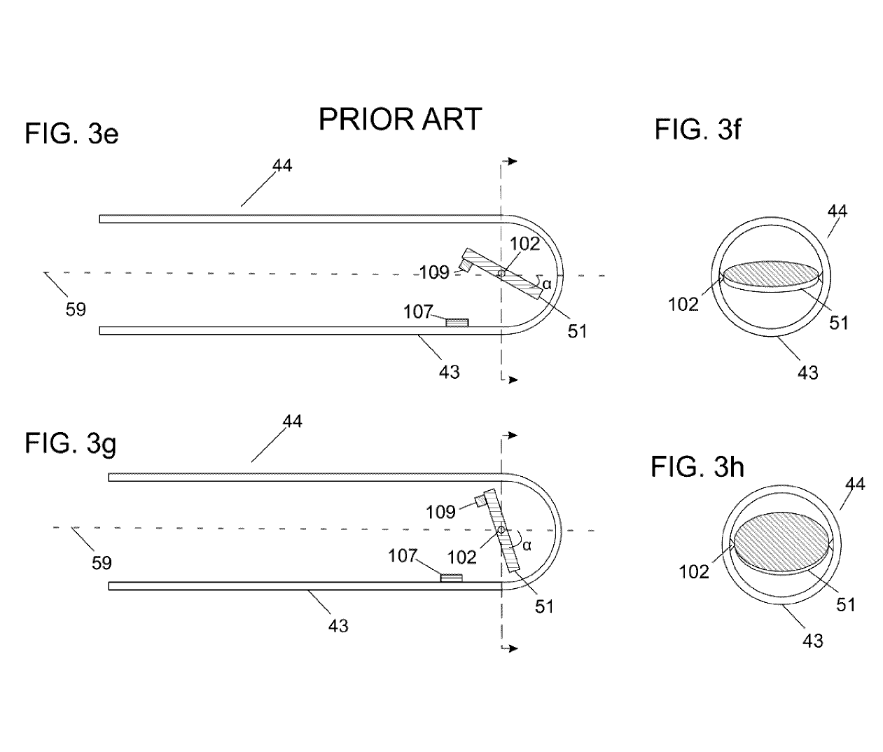

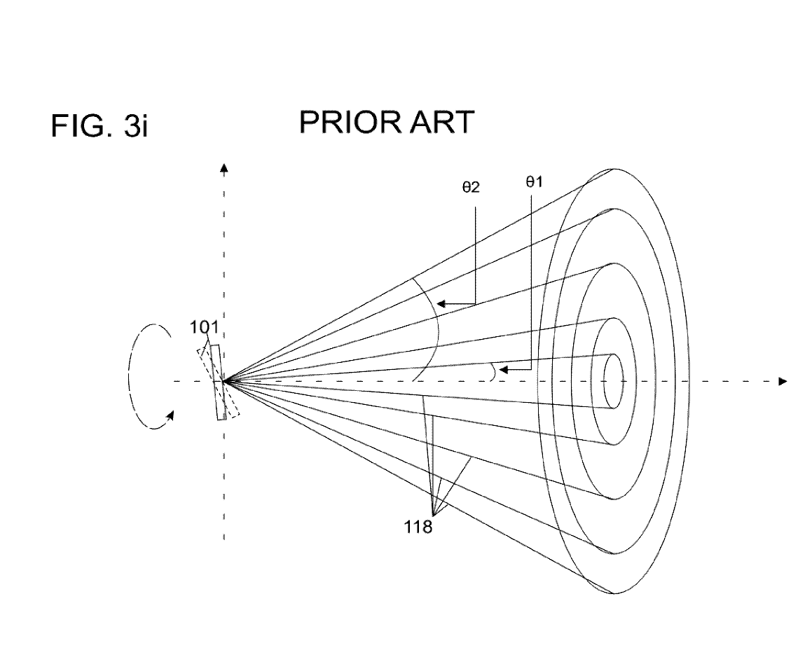

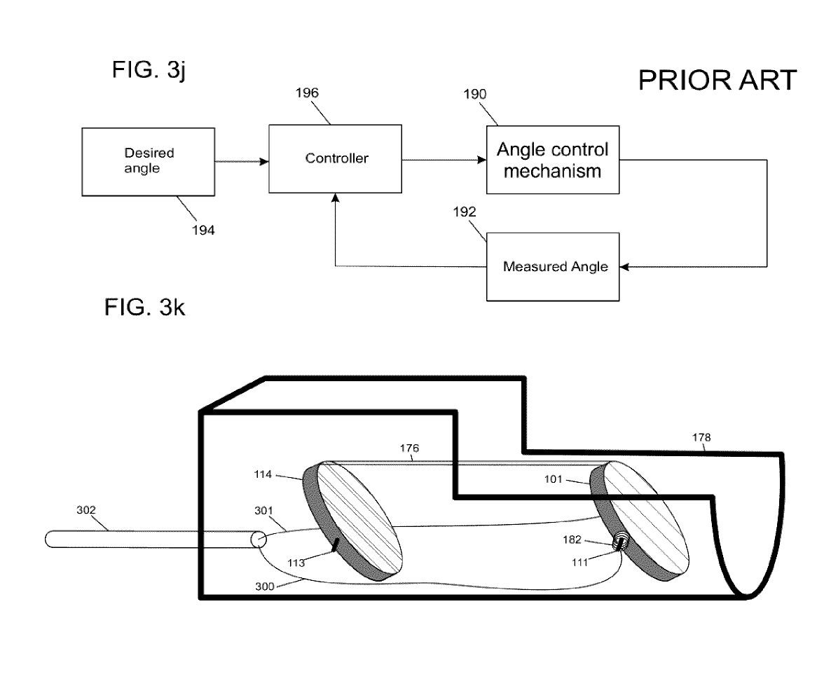

FIGS. 3a-d show an example imaging probe that employs a tiltable member for scanning an imaging beam. FIG. 3a shows a perspective cutaway drawing of the distal region of an imaging probe 44 that relies on centripetal force to generate the change in tilt angle of the tiltable member 51. The imaging probe 44, which includes a sheath 43 for isolation from bodily fluids and cavities, includes tiltable member 51, which may be housed within an imaging assembly, as shown in FIG. 2B.

Tiltable member 51 is mounted on pins 102, about which tiltable member 51 is able to pivot and is bias towards its starting position with the use of a restoring force. As imaging conduit and assembly (not shown) are rotated about longitudinal axis 59 at a slow rate (indicated by arcing hatched arrow 61), the angle α subtended between longitudinal axis 59 and tiltable member 51 is relatively small. A cutaway perspective cross-sectional view of FIG. 3a is shown in FIG. 3b. FIG. 3c shows a similar drawing of the distal region of imaging probe 44 as shown in FIG. 3a, except with imaging conduit 46 being rotated at a faster rate (indicated by arcing hatched arrow 63) than in FIG. 3a. Centripetal force causes tiltable member 51 to tilt such that there is an increase in the angle α subtended between the longitudinal axis of the catheter and the tiltable member 51. FIG. 3d is a cutaway perspective cross-sectional view from FIG. 3c.

FIG. 3e shows a perspective cutaway drawing of the distal region of a related imaging probe 44 that relies on the use of dynamically controlled magnetic fields to change the deflection angle of tiltable member 51. Imaging probe 44, which may include a sheath 43 for some degree of isolation from bodily fluids and cavities, includes tiltable member 51 comprising part of the imaging assembly 50. Tiltable member 51 is mounted on pins 102, about which the tiltable member 51 is free to pivot. Mounted on the tiltable member 51 is a magnetically influenced element 109 that can be either attracted or repulsed by a magnetic field. For example, it may be a ferromagnetic component, or a permanent magnetic component. Element 109 may integrally be part of tiltable member 51, such as if all or a portion of element 109 is made of either a ferromagnetic or magnetic substrate. An electromagnetic component 107 is also placed at a position separate from the tiltable member 51. The electromagnetic component can be controlled to produce attractive or repulsive forces relative to magnetically influenced component 109. In so doing, the angle α subtended between the longitudinal axis 59 of the catheter and the tiltable member can be adjusted as desired. Furthermore, similar imaging probes may be conceived that involve interchanging the position of the electromagnetic component 107 and magnetically influenced component 109, or using two electromagnets instead of an electromagnet and a magnetically influenced component. A cutaway perspective cross-sectional view of FIG. 3e is shown in FIG. 3f.

FIG. 3g shows a similar drawing of the distal region of imaging probe 44 as shown in FIG. 3e, except with a repulsive sequence applied to electromagnet 107 such that the angle α subtended by tiltable member 51 is increased. FIG. 3h is a cutaway perspective cross-sectional view from FIG. 3g.

Tiltable member 51 may be an ultrasonic transducer, such as an ultrasound transducer used for producing B-scan ultrasound images. Another embodiment includes an ultrasound transducer mounted on a tiltable member.

FIG. 3i shows an example of a potential scanning pattern for generating ultrasound images. In this case, the tiltable member is an ultrasound imaging transducer 101. As imaging conduit and assembly (not shown) are rotated at a constant rate, an image is generated along a surface that approximates a cone. As the rate of rotation is changed, centripetal force causes the angle subtended between the longitudinal axis of the catheter and ultrasound imaging transducer 101 to change resulting in a series of concentric imaging cones 118 for different rotational speeds. The angle subtended between the longitudinal axis of the catheter and an axis normal to ultrasonic imaging transducer 101 will be referred to as the “imaging angle”. In this case, the transducer begins with a relatively small imaging angle θ1 implying a fast rate of rotational speed. As the rotational speed is reduced, the imaging angle is increased to θ2.

In some embodiments, a mechanism may be provided for detecting the tilt angle of the tiltable member. A number of example implementations are described in PCT Patent Application No. PCT/CA2012/050057, titled “ULTRASONIC PROBE WITH ULTRASONIC TRANSDUCERS ADDRESSABLE ON COMMON ELECTRICAL CHANNEL”, which is incorporated herein by reference in its entirety. As shown in FIG. 3J, the imaging angle may be employed for feedback in a control system. A desired angle 194 and the measured angle 192 are provided as inputs to controller 196, and the output of controller 196 is provided to angle control mechanism 190. A variety of control methods and algorithms known in the art may be employed, including, but not limited to, PID and fuzzy logic controllers.

In order to cause the imaging angle to return to a stable position in the absence of rotation, a restoring mechanism can be used as shown in FIG. 3k. Here, the primary movable member 101 is connected to a secondary movable member 114 using a mechanical coupler 176, allowing the two members 101 and 114 to move synchronously. All components are housed within a shell 178. One or more springs 182 are connected between the movable member 101 and the shell 178. The springs may be torsion springs, linear springs, or a cantilever spring. The movable members 101 and 114 are pivotally supported by around pins 111 and 113 respectively. This spring 182 provides a force to restore the member 101 to the side viewing position in the absence of adequate rotational force to overcome the restoring force provided by spring 182. In addition to adding a mechanical restoring force, the torsional springs may also be formed, at least in part, from an electrically conductive material, such as stainless steel, beryllium copper, copper, silver, titanium, gold, platinum, palladium, rhenium, tungsten, nickel, cobalt, alloys that include one or more of these metals and many other metals and their alloys can be used to provide electrical connections. Here, spring 182 is in electrical communication with conductor 300. Conductor 301 makes a similar connection to the opposite side of movable member 101 (not shown).

For clarity, “rotatable” or “rotating” components refer to components that rotate when actuated with a rotating mechanism. An example of a rotatable component is a torque cable (described and shown below), at least a portion of which lies within an external sheath of catheter 100 and is able to rotate independent of the external sheath. “Non-rotating” components refer to components that do not rotate with the rotatable shaft, but may nonetheless be rotated, such as under manual manipulation of the catheter's outer housing or external sheath.

The configuration shown in the above figures is one of many examples of medical probes. Other configurations and types of imaging devices can be located in the distal region of the probe. Probes which employ various ultrasonography techniques such as elastography, compression ultrasonography, and Doppler ultrasonography are relevant. US patent application 20080177183 (Courtney et al), incorporated herein by reference in its entirety, describes embodiments for combined ultrasound and optical imaging probes.

Imaging catheters, such as intravascular and intracardiac ultrasound catheters, typically require the catheter body to be purged of air prior to operation. The purging is performed to support the efficient propagation, within the catheter body, of imaging energy generated or detected by one or more internal transducers. For example, in the use of commercially available mechanical intravascular ultrasound (IVUS) catheters, it is common to have to purge air from the main lumen of the IVUS catheter by replacing it with a media such as saline, water or another medium that provides better acoustic coupling between the ultrasound transducer and the wall of the catheter, which in turn provides acoustic coupling to the surrounding environs that are imaged with IVUS, such as blood and vascular walls.

The fluid is commonly introduced into the catheter by a procedure referred to as “flushing” the catheter, where fluid is injected into the catheter via a port at the proximal region. This fluid, which is typically a liquid such as saline or sterile water, travels along the length of the main lumen of the catheter and purges undesired air out of a port near their distal end.

Other catheters do not support flushing of the catheter through ports available outside the body. Such catheters typically require manual injection of a fluid coupling medium to the distal tip of the catheter via a hypodermic needle attached to a syringe. For example, some intracardiac echocardiography catheters have only a single port. The UltraICE™ catheter by Boston Scientific has a foam port at its distal end that acts as both an influx and efflux port. A user of the catheter vents air out from the distal portion of the catheter by inserting a needle through a foam member into the distal chamber of the UltraICE catheter. The user then injects a displacing fluid (typically sterile water) into the distal chamber. As the displacing fluid enters the distal chamber, air escapes through the porous foam member. In one example implementation, a catheter may be provided in a pre-filled state, without having an external port.

Other intravascular and intracardiac imaging catheters are also under development. US Patent Publication No. 20080177138 (Courtney et al.), which is incorporated herein by reference in its entirety, describes embodiments for forward-looking imaging catheters. In some embodiments, it will be preferable for the ultrasound transducer to be located near the distal region of a catheter.

In some embodiments, it will be preferable for the distal tip of the catheter to not be in fluid communication with blood. US Patent Publication No. 20130023770, titled “MEDICAL PROBE WITH FLUID ROTARY JOINT”, and which is incorporated herein by reference in its entirety, describes embodiments of imaging catheters where the influx and efflux ports for flushing are located near the proximal region of the catheter, outside of the body and blood-filled vasculature.

It is desirable that flushing is a safe, simple, quick and effective procedure. To achieve this, facilitating the removal of air or other media, that are not flushing fluid, from inside the catheter, is critical. Air bubbles are hydrophobic and often occur within catheters for a number of reasons. Some materials used in catheter components are inherently hydrophobic in nature and initially in contact with air. When flushing fluid is introduced, air remains attached to the surface in some areas, due to attractive forces between a hydrophobic surface and air bubble. This adhesion is further encouraged by the behavior of the water molecules which tightly coalesce and rearrange around the air bubble, entrapping it, and isolating it from the rest of the hydrophilic solution. These interactions are favorable as they lower the total energy of the system, bringing it to equilibrium.

As such, it may be difficult to remove air bubbles via flushing. This can especially occur at regions or surfaces where convective flow of the flushing fluid is not sufficient to urge bubbles to be displaced. The flushing act does not create adequate local forces to overcome the forces that cause the bubble to adhere to a surface. On surfaces where imperfections and uneven textures exist, the hydrophobic nature dominates, even on the micrometer and nanometer scales, creating a point of higher contact angle. These regions are prone to the manifestation of air bubbles and it becomes energetically unfavorable to displace bubbles with flushing fluid. Bubbles can also be created through degasification, where gasses dissolved in the fluid are released from the solution, such as during a change in pressure of the solution. Furthermore, undissolved bubbles which individually may not be problematic can coalesce to form larger bubbles.

In catheters and other devices where a distal flushing efflux port is available within the body, the existence of media different from the flushing fluid can enter the body and have adverse effects. For example, an air bubble existing on the inner lumen of the catheter might be urged into the body by the act of flushing and could act as an embolus. In many applications, it is also desirable to fluidly isolate inner portions of catheters from the anatomic environment in which they are used. For example, if blood enters the inner lumen of an imaging catheter, it may degrade image quality by interfering with sensitive acoustic, electrical or mechanical mechanisms within the catheter.

In minimally invasive imaging devices, air bubbles are known to have a negative impact on imaging quality. Specifically, in the intravascular ultrasound catheters and intracardiac echocardiography catheters, acoustic waves used for imaging are reflected and/or scattered when they encounter a change in acoustic impedance along the propagation path. In addition, bubbles can interfere with the fine mechanical motion of imaging components. Optical assessment modalities can also suffer from inhomogeneities in the media through which optical beams travel. Optical coherence tomography (OCT) is one such imaging modality.

As an alternative to flushing via a proximal port and allowing fluid to exit via an efflux port, some catheters have been designed with an inner lumen as part of the imaging catheter to deliver fluid to the distal region of the catheter, allowing the fluid to “backfill” the outer lumen of the catheter. Alternatively, the separate lumen can used as a venting lumen, where the fluid is introduced via the inner lumen, and the outer lumen allows air to escape. However, these approaches may still have inadequate capabilities to remove air bubbles with ease. In fact, in catheters with a closed distal portion and no distal flush port, flushing media that is directed toward the distal inner region of the catheter in a proximal to distal direction has to change directions in a distal to proximal direction in order to exit the catheter. This can create regions where the flow velocities within the distal portion of the catheter are much lower in magnitude, and thus less able to urge bubbles from this area, than they would be along the rest of the length of the catheter.

Several solutions exist for removing bubbles, including increasing the flow rate through the regions in which bubbles reside. However, this approach can be difficult to implement, as it may require higher injection pressures than are not easily achieved manually by users with a common syringe size (such as a 2-60 cc syringe). Furthermore, higher injection pressures place additional mechanical and geometric demands on catheter components, which are often advantageously designed to have minimal feature sizes to minimize trauma during their use. Another frequently used approach is for the physician to minimize the presence of air bubbles during operation by flushing liquid prior to insertion of the catheter into the vasculature. This approach often fails to eliminate the bubbles entirely as the length of the catheter is relatively long and bubbles can come out of hidden regions or out of solution during use of the imaging catheter.

Embodiments disclosed below provide a medical probe, where one or more regions of its inner surfaces exhibit hydrophilic properties. In some embodiments, one or more internal surfaces of the medical probe are modified to become hydrophilic. Embodiments described herein enable the urging of air bubbles away from inner surfaces of internal surfaces or components within the probe, thus providing advantages and benefits related to system performance, ease of use, and safety.

In some embodiments, the present disclosure describes devices that employ hydrophilicity to facilitate the urging of bubbles from forming on inner surfaces of an imaging probe, as well as easing the elimination of bubbles that have formed on these surfaces or are free floating. In some embodiments, prior to use, the medical probe does not contain flushing fluid and thus the inner surfaces of the probe are initially in contact with air.

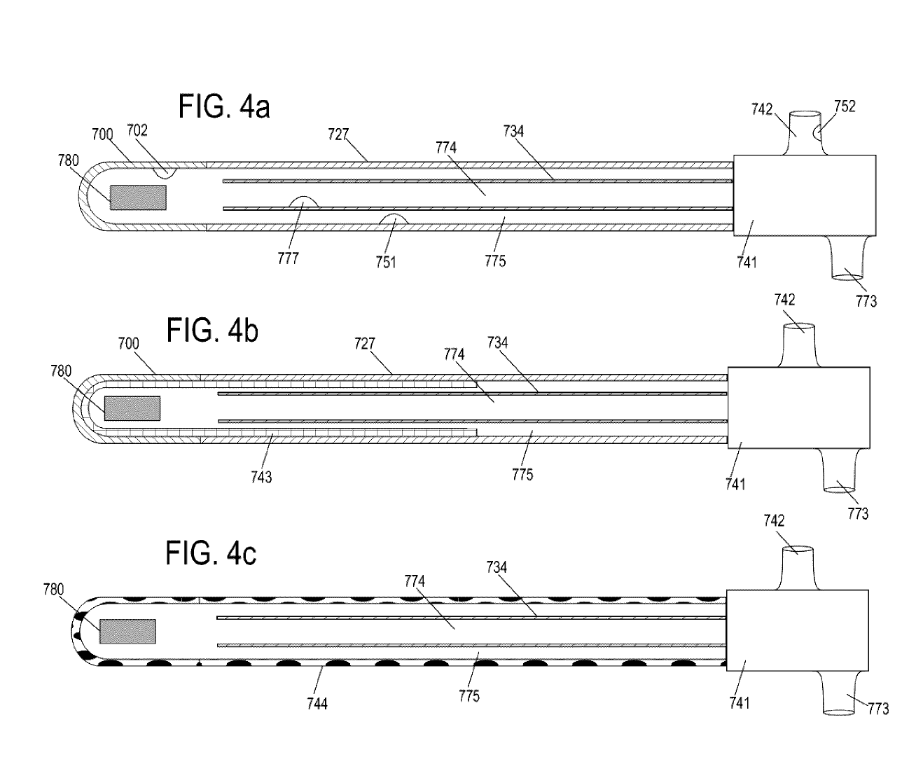

FIG. 4(a) illustrates an example of a medical probe, comprising a functional device 780, which could be an imaging device, therapeutic device, or another kind of device, which could be included in a catheter (supported, for example, by an internal shaft, torque cable, or other structure, which is not shown in the figure). The probe also consists of a distal dome 700 bonded to a catheter sheath 727 containing an inner conduit 734. There is an inner lumen 774 within the inner conduit 734 and the outer lumen of the catheter 775 is the area between the inner conduit 734 and catheter sheath 727, defining an internal fluidic path. The proximal ends of the sheath 727 and inner conduit 734 are connected to the proximal connector 741, which consists of an influx port 742 and an efflux port 773. In such a configuration, flushing occurs by inserting fluid into the influx port 742 of the proximal connector 741 which fills the inner lumen 774 and delivers fluid to the distal dome 700. The fluid then traverses around the inner conduit 734 and backfills the outer lumen of the catheter 775. US Patent Publication No. 20130023770, titled “MEDICAL PROBE WITH FLUID ROTARY JOINT”, and which is incorporated herein by reference in its entirety, describes embodiments of imaging catheters which include an inner and outer lumen, utilizing such a flushing method.

The distal imaging assembly is not included as this figure is provided for illustrative purposes only. Different types of imaging assemblies using optical components or combined ultrasound and optical components could be included within the distal region and examples are shown in some of the later figures.

In FIG. 4(a), air bubbles 702, 751, 777 and 752 are shown adhering to various hydrophobic internal surfaces, including the inner surfaces of the distal dome 700, of catheter sheath 727, of inner conduit 734, and of proximal influx flush port 742 of the proximal connector 741, respectively. Although this embodiment shows only one entry and one exit fluid port, the medical probe may be in fluidic communication with one or more external fluid ports used for introducing and removing a liquid thereto. The air bubbles adhere to these internal surfaces due to the hydrophobic nature of these inner surfaces.

When such internal surfaces are hydrophilic instead of hydrophobic, it becomes energetically favorable to wet the surface and disengage air bubbles. Without being limited to theory, it is believed that hydrophilic surfaces are ionic in nature and attract aqueous and polar substances via means of dynamic hydrogen bonding. When an aqueous flushing fluid is introduced, the air adhering to a hydrophilic surface is displaced with water molecules, as this action results in a lowered surface tension. If an air bubble is introduced into a hydrophilic region from another region of the catheter, such as an area that is not hydrophilic, it will be energetically unfavorable for the introduced bubble to adhere to the created hydrophilic surfaces and will be displaced when flushed.

In some embodiments, one or more internal surfaces of a medical probe may be configured to exhibit hydrophilicity by forming at least a portion of the medical probe from a material that is intrinsically hydrophilic in nature. An extrudable hydrophilic polymer such as PEBAX MV1074 SA 01 MED from Arkema is one example of an inherently hydrophilic material. Such materials have high swelling ratios and can be disadvantageous in some medical applications discussed in the present disclosure.

In some of the embodiments described herein, one or more internal surfaces of a medical probe may be modified to exhibit hydrophilicity. One example technique for modifying a surface such that it becomes hydrophilic is to apply a hydrophilic layer. For example, in the embodiment shown in FIG. 4(b), an inner surface of the medical probe is modified to be hydrophilic by adding a hydrophilic layer 743. In this embodiment, the coating is applied to a portion of the inner surface of the hollow shaft, but it will be understood that the coating can be applied to as large of an area as deemed appropriate.

In some example implementations, a hydrophilic coating may be a polymer based coating. Non-limiting examples of suitable coatings contain ingredients such as: polyethylene oxide, a poly acrylate base coat with a polyurethane top coat, polyurethane resin, polyhydroxyethyl methacrylate, and polyvinylpyrrolidone. Other examples of coatings include ceramic-based coatings, which mitigate the swell issues that may result from the use of polymeric coatings. Example ceramic-based coatings may contain alumina, titanium nitride, silver oxide, zirconia, zinc oxide, titanium dioxide, or copper oxide. Heat and UV are the two main curing techniques used for hydrophilic coatings, but others are available. Curing temperatures often range from 40-60 degrees Celsius for temperature sensitive materials and 80-100 degrees Celsius for others. Curing times typically range from 60-480 minutes depending on the application technique and coating used.

Several processes can be used for the application of a hydrophilic coating to a surface. Non-limiting examples of such methods include spin coating, spray coating, dip coating, injection, ultrasonic atomization, application with a sponge or roller, vacuum deposition, and ink-jet printing.

In one example implementation, plasma-deposited coatings may be used to alter the hydrophilicity of a surface. For example, component material can undergo plasma treatments in a radiofrequency discharge of nitrogen, argon, or helium to deposit ultra-thin layers using plasma. Wettability is improved through this technique by the generation of oxygen functionalities. A silicon oxide layer is one example of a stable hydrophilic surface which can be created using this technique.

A medical probe having an internal hydrophilic surface extending over a limited internal region can be fabricated by applying a hydrophilic layer to one or more components (such as the internal surface of a sheath) after the component has been extruded into its desirable shape, but prior to assembly of the medical probe.

In some example implementations in which one or more components are coated with a hydrophilic layer prior to assembly of the medical probe, one or more regions of components require additional processing prior to assembly of the medical probe in order to remove a portion of the coating. For example, some catheters are manufactured by bonding a distal dome to the distal portion of the sheath of a catheter. This is commonly achieved via methods such as adhesive bonding, UV curing, thermal bonding, or RF bonding. In such cases, it may be beneficial or important to ensure that bonding areas are substantially free of coatings such that when the dome and sheath are bonded, the coating does not interfere with the bonding process nor affect the created bond. Keeping these areas free of coating will also preserve the effectiveness of the coating because bonding processes may alter coating properties.

In order to achieve uncoated areas, a hydrophilic layer can be applied to all surfaces of the component of interest and then removed from undesired areas prior to bonding. This can be achieved, for example, through mechanical abrasion. In one example, mechanical abrasion may be performed using a method in which small diameter endmills or engraving bits used for patterning printed circuit boards are loaded into a milling machine. For example, using a 4 axis Computer Numerical Controller, the coating can be removed from the areas of interest. In another example implementation, strong solvents such as acetone may be used to degrade, dissolve and remove the coating off the desired areas. The solvent selected will be dependent on the composition of the hydrophilic coating.

Alternatively, masking techniques can be used to coat selected areas of a surface while blocking other areas. One example masking technique is to cover areas which are to be coating-free with medical grade tape and then remove the tape after the coating process is complete. For example, one could apply tape to the inside of the sheath by measuring and marking the areas which need to be masked from the coating. Long micro tweezers or other such micro instruments can then be used to insert the tape into the extrusion and place the tape on the desired areas. For better visibility, the masking and coating processes would be executed prior to assembling the catheter. Also the extrusions can be placed under a microscope to ensure the tape is placed at the marked areas. The transparency of the extrusions will allow for better visibility. Removal of the tape could be performed using the same instruments. Another example of a masking technique is to create a microsphere polymeric mask over component areas that are desired to be free of hydrophilic coating. This can be done by drop-coating the areas with polystyrene nanospheres and allowing to it to dry. During the drying process, a close-packed hexagonal monolayer is formed which protects the substrate during the coating process. After the process is complete, the components can be sonicated (for example, for time duration of 3-5 minutes) in ethanol to remove the polystyrene mask. To use this masking technique for a cylindrical sheath, one would have to create sheath segments of limited length, such that each segment consists of one masked area and one coated area. The limited length would make areas accessible for drop coating one side with polystyrene nanosphreres and coating the other side with hydrophilic coating. After removal of the polystyrene masks, the segments would then be bonded together to create the final length of the catheter.

In addition to the techniques mentioned above, other methods may be employed when the component material to be coated is other than a polymer. For example, chemical treatments such as liquid-phase treatments may be used to chemically alter a portion of the inner surface of a catheter. For example, immersing the component material into an ethanol solution over a period of time could increase the hydrophilic nature of the surface directly in contact with ethanol. For silicones, such as polydimethylsiloxane (PDMS), one can use potassium hydroxide solution to increase the hydrophilicity of the surface.

In another example implementation, radiation methods such as ionizing radiation may be employed for local surface treatments in order to achieve hydrophilicity. For example, in the case of selected plastics, the processing parameters of chemical and radiation treatments may be tightly controlled such that a hydrophilic surface is obtained without causing polymer degradation. Examples of such plastics include, but are not limited to, polystyrene, polysulfone, polyurethane, polyimide, and allyl diglycol carbonate.

In some example implementations, an electrically insulating hydrophilic layer can be provided such that undesired shorting between electrical components within the catheter and shock to the patient do not occur. One example of such a coating is hydrophilic Parylene.

Various embodiments described herein refer to an “operational wavelength” of a transducer. The term “operational wavelength” may be defined as described below. In a piezoelectric ultrasound transducer stack, the thickness of the active layer is often designed to be substantially less than the width of the active layer (typically 1/10^{th} the size or smaller). This is done to separate the frequency of the fundamental thickness resonant mode of the layer from any lateral resonance mode. Within any propagating material or medium (such as conductive silver epoxy or human tissue) the propagating waveform will have a fundamental wavelength that is related to the frequency of the fundamental resonance mode through the speed of sound of the material or medium, according to the relation: Wavelength=speed of sound/frequency. In some embodiments, the operational wavelength may be this fundamental design wavelength.

In real transducers, materials are not perfect (ideal) resonators and therefore the fundamental frequency is actually a band of excited frequencies that can be characterized by a center frequency and a bandwidth of excited frequencies. Matching layers and backing layers are added to effectively couple as much of the resonant energy out the front face of the transducer stack and into the propagating medium in as short a time as possible. This will result in yet a broader frequency response of the stack, (i.e. broader bandwidth of excited frequencies) allowing for the transducer stack to more closely replicate an ultrasound pulse response waveform from a short excitation transmit signal (say a single cycle waveform), as well as from a more narrow band excitation pulse such as a tone burst of several cycles in duration. Fabrication tolerances can also result in deviations of the time and frequency response of the transducer. In some embodiments, the operational wavelength associated with the transducer may be the wavelength within the frequency response of the stack, such as the center wavelength. For example, in some embodiments, the operational wavelength associated with the transducer may include any wavelength within this combined design, excitation pulse, and fabrication tolerance dependent bandwidth.

In some embodiments, the medical probe is an ultrasonic imaging probe having a hydrophilic coating layer applied to one or more internal surfaces, where the properties of the coating layer are selected such that it has desirable acoustic properties, such as speed of sound, density, acoustic attenuation, acoustic impedance and layer thickness. Such properties may influence signal transmission efficiency and beam shape, preferably in a favorable manner.

If the hydrophilic layer has similar acoustic properties to water or saline, which are frequently used as media to couple the transducer to the imaging probe sheath, which in turn acoustically couples an ultrasound imaging probe to the surrounding anatomy to be imaged, the hydrophilic layer can act to enhance transmission.

In one example implementation, the coating layer may be used as a matching layer of the ultrasound transducer residing within the imaging catheter, and may be applied to any internal surface through which imaging energy is transmitted, and which is in flow communication with the fluid path. Such surfaces are referred to as “imaging surfaces”, and may include internal surfaces such as, but not limited to, the internal surface of the sheath and a surface of an imaging transducer. To function as an ideal matching layer (which is not required in many of the embodiments described herein) the matching layer should meet two criteria, one of which is to have an acoustic impedance equal to the geometric mean of the acoustic impedances of the matching layer's adjacent media. The second criterion is that the matching layer should have a thickness approximately equal to a quarter of an operational wavelength of acoustic energy generated by the ultrasound transducer as it travels through the coating layer.

In one example implementation, the operational wavelength associated with a broad band pulse may equal the wavelength within the coating that corresponds to any frequency falling within the 6 dB bandwidth of the center frequency of the pulse.

In one example, depending on the material of choice, the matching layer applied to an imaging surface can have a thickness falling in the range of approximately 0.23 to 0.27 of an operational wavelength of acoustic energy generated by the ultrasound transducer as it travels through the coating layer, and still act as an effective matching layer.

In some embodiments, a hydrophilic coating is formed by one or more polymer layers.

The thickness of the coating can be tuned by controlling the volume swell ratio of the coating (or one or more the layers of a multi-layer coating). For example, the swell ratio can be controlled by increasing or decreasing the cross-linking of one or more layers. Cross-linking can be controlled by selecting the type, temperature, concentration and dwell times of one or more cross-linking additives.

One example of a suitable cross-linking additive is sulfur, which is added in a vulcanization chemical process and promotes the formation of crosslinks between individual polymer chains, thus lowering the swell ratio. As a result, the acoustic impedance of the layer would also be affected due to changes in density and thus can be tuned as desired in addition to optimizing the layer thickness.

In some cases, to achieve optimized transmission, the thickness may not fall within the workable range of 0.23 to 0.27 of the transducer operational wavelength. Here the acoustic impedance of the layer can be tuned in order to minimize reflections. In one example implementation, the hydrophilic coating can be selected to reduce the impedance mismatch between a liquid residing or flowing within the ultrasonic imaging catheter (e.g. a flushing liquid) and one or more components of the ultrasonic imaging probe through which ultrasonic imaging energy propagates. The hydrophilic coating can be applied on an internal surface of a catheter sheath and the impedance of the hydrophilic coating may be selected to lie between the impedance of the liquid within the imaging catheter and the impedance of the sheath (for example, the impedance of the hydrophilic coating may be the be the geometric mean of the impedance of the liquid within the imaging catheter and the impedance of the sheath).

In one example embodiment, the coated component is the dome of an ultrasound catheter, then the media on either side of the matching layer are the liquid and dome material. For example for a 40 MHz transducer, hydrophilic Parylene may be a suitable coating that has an acoustic impedance of 2.7 Mrayls and can act to reduce the impedance mismatch between a water flushing fluid which has an acoustic impedance of 1.48 Mrayls and a dome/sheath material formed from PEBAX, where the acoustic impedance varies depending on the grade and thickness.

There are examples where a matching layer has an acoustic impedance that does not fall at or near the geometric mean of the acoustic impedances of the two interfacing media. In some of these examples, the effectiveness of the matching layer may still be sufficient given the application. In other cases, different properties of the matching layer can be tuned. For a coating layer, the thickness can be designed to minimize reflections. An example being that the matching coating layer can be selected to be extremely thin, having a thickness equal to or less than approximately one tenth of the transducer operational wavelength. Such a configuration implies that the coating layer is acoustically transparent and the presence of an acoustic mismatch does not have an effect on the performance. For example, for a 10 MHz signal, where the operational wavelength of acoustic energy generated by the ultrasound transducer is approximately 220 micron in Parylene, the coating layer thickness could be selected to be less than 22 microns.

Common internal flushing fluids used are: saline, sterile water, tap water, deionized water, lactated Ringer's solution, and phosphate buffered saline. Examples of the materials which may be used for dome and sheaths include: PEBAX, low density polyethylene and nylon. The coating can be selected depending on which combination of flushing fluid and dome/sheath materials are used.

In some embodiments, one or more geometric or spatial aspects of the coating (e.g. thickness and swelling ratio) and/or mechanical properties (e.g. abrasion resistance) of the coating, can be tuned to improve the mechanical functionality of the imaging device by increasing lubricity and lowering the friction of mechanical components such as tilting transducers, torque cables, and springs. An additional advantage of lowering the friction within the catheter is the reduction of non-uniform rotational distortion (NURD) that may occur due to the rotating transducer. Applied hydrophilic layers can be made more lubricious by mixing ingredients such as Teflon-like fluoropolymers in the coatings formulation. To increase the abrasion resistance of a coating, the degree of crosslinking can be increased, for example, to make it more rigid and durable. As a result this would lower any friction created by the mechanical components within the catheter.

An alternative method of making a surface hydrophilic is depicted in the example embodiment presented in FIG. 4(c), where one or more hydrophilic additives are added directly to the component material during its fabrication such that the material becomes inherently hydrophilic. As a result, the impregnated inner surface 744 possesses enhanced surface wettability and promotes the adhesion of water molecules.

In some embodiments, only a portion of an inner surface of the hollow shaft is modified with additives, but this can be extended to as large of an area as deemed appropriate.

In one example implementation, a polymer material with a hydrophilic surface may be formed by adding one or more oligomeric additives to a polymer melt while polymerization is taking place. In such a case, the oligomeric additive chains may then adhere to host polymeric chains and migrate to surfaces, generating surface reactivity without modifying bulk material properties. A few example hydrophilic additives include acidic groups such as polyvinyl alcohol, sulfonate, hydroxyl, mercapton, carboxylic, or carbonamide. It is to be understood that such additives, and their properties, should be selected such that they are compatible with the base polymer and achieve the desired properties from the final formulation.

For the fabrication of such a medical probe, hydrophilic additives are mixed into the formulation during polymerization of base component material. Mixing occurs prior to the extrusion of components into its desired formation and prior to assembly of the medical probe. Since all polymer ingredients in a formulation are exposed to hydrophilic additives, all the surfaces of a component will have enhanced wettability. As such, separate formulations must be created for surfaces which are not to be hydrophilic. The different hydrophilic and non-hydrophilic segments can then get bonded to each other to create the final areas of hydrophilicity and hydrophobicity along the length of the device.

In the case in which the medical probe is an imaging probe, hydrophilic additives may be selected such that the final component material formulation is acoustically and/or optically transparent such that they have minimal to no interference with device performance. In some embodiments, one or more properties of an additive may be controlled in order to achieve desired acoustic and optical properties. Additive properties which can be varied include, but are not limited to: molecular weight, hydrocarbon chain length, hydrocarbon chain configuration, and hydrophilic-lipophilic balance. In some embodiments, the concentration and weight percent of the hydrophilic additive can be altered such that the final impregnated component material has desirable acoustic properties.

One pertinent advantage of an embodiment in which the surface of the component material becomes inherently hydrophilic is that the hydrophilicity is permanent. In embodiments in which a hydrophilic layer is used, there is a possibility that the layer may wear down, be scratched off, disintegrate, or otherwise degrade.

A potential benefit of mixing additives into the component material (e.g. the dome and sheath material), such that the hydrophilicity is achieved on both the inner and outer surfaces of the component, is the additional lubricity induced on the external surfaces of the catheter. This property allows for ease of insertion of the medical probe into the vasculature of interest, and also enhances the maneuverability of the medical probe. In addition, lubricity contributes to patient safety by lower the possibility of causing vascular damage during use.

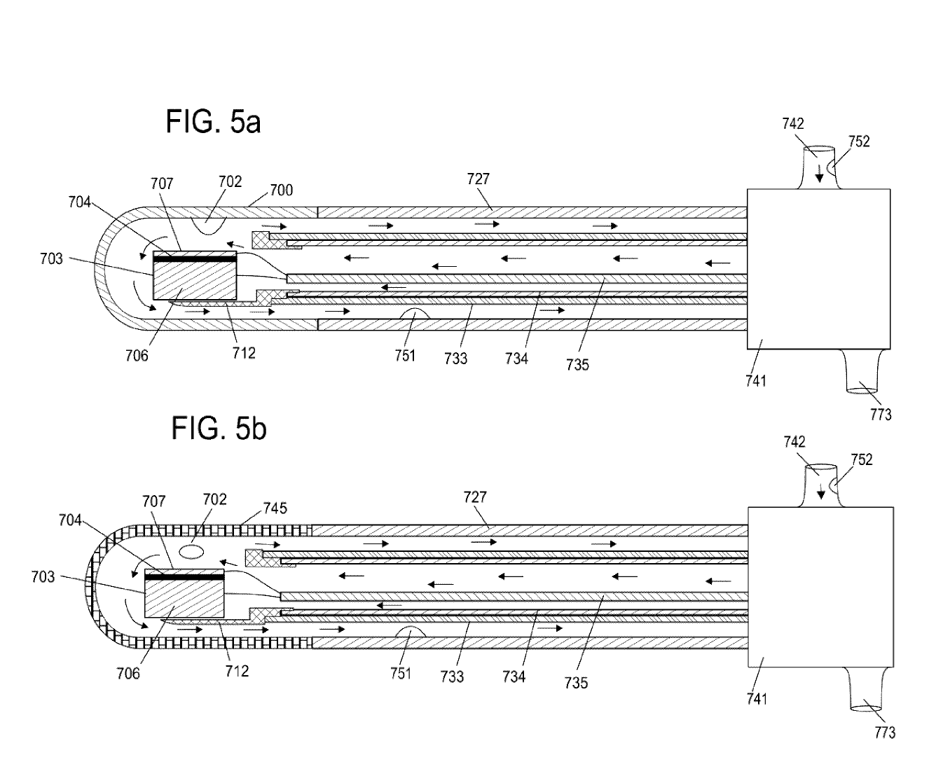

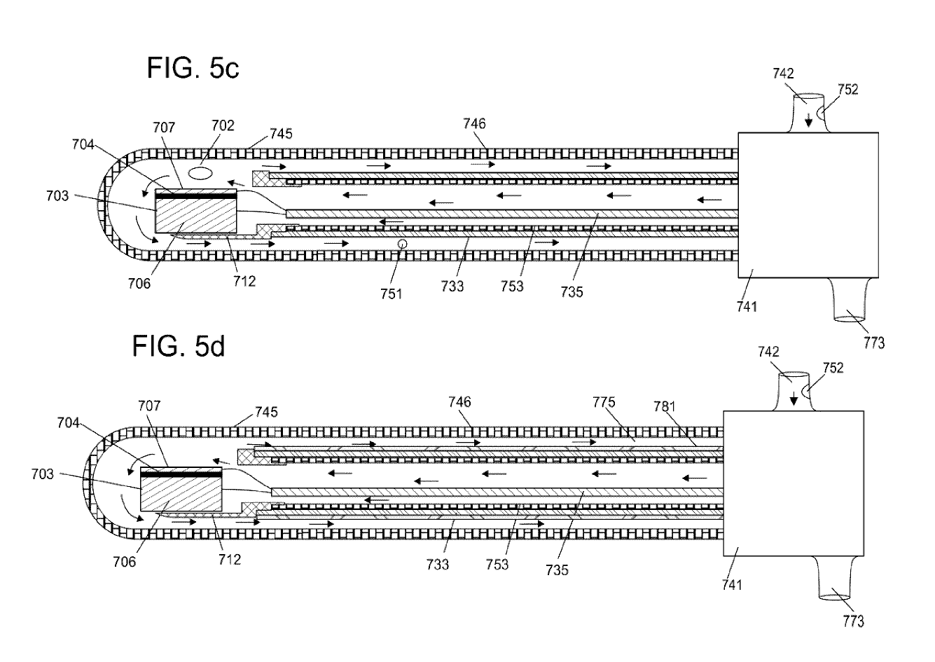

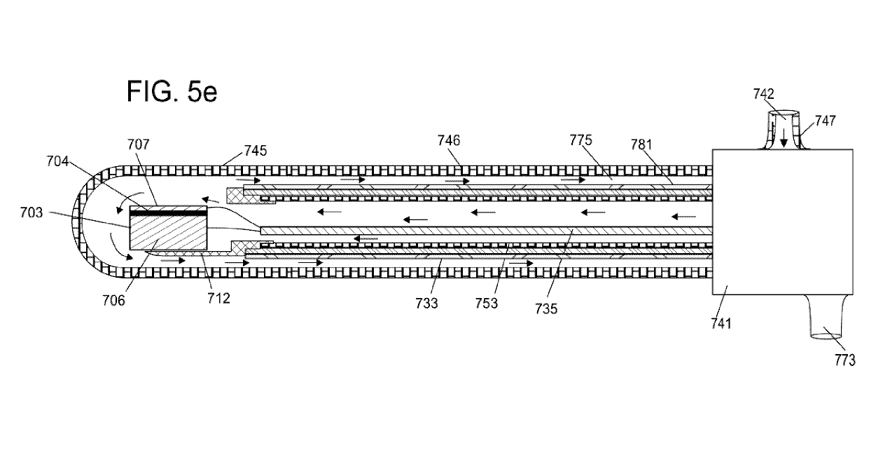

FIG. 5(a) illustrates the problems associated with untreated surfaces within an example ultrasonic imaging probe. The ultrasonic transducer 703 which sits within the imaging assembly housing 712 consists of the transducer conductive backing layer 706, acoustic substrate electroded on both sides (piezoelectric) 704, and a transducer conductive matching layer 707. In this configuration, the transducer is at rest and not under operation. Under operation the transducer would be side viewing as well as forward viewing.

The flushing fluid flow path is depicted with the fluid in-fluxing into the proximal influx port 742, moving into the inner conduit 734 around the electrical cable (consisting of one or more conductors) 735, in a proximal to distal direction, towards the untreated distal dome 700 of the catheter. The flushing fluid then effluxes in a distal to proximal direction, around the inner conduit 734 and torque cable 733. The untreated inner surfaces adhere an air bubble 702 on the distal dome 700, an air bubble 751 on the catheter sheath 727, and an air bubble 752 on the proximal influx flush port 742 of the proximal connector 741.

In the example embodiment shown in FIG. 5(b), distal dome 745 has a hydrophilic inner surface such that air bubble 702 no longer adheres to the surface and is flushed away from this area. As noted above, in some embodiments, the inner surface of distal dome 745 may be made hydrophilic (e.g. modified to be hydrophilic) by, for example, applying a hydrophilic layer, or by impregnating component material with hydrophilic additives. The hydrophilic surface may be imparted onto distal dome 745 before it is bonded onto the sheath. As such, the device does not have any bubbles along the inner surface that would obstruct the propagation of ultrasound signals from the transducer 703. It is evident that air bubbles 751 and 752 still adhere to the unmodified surfaces.

In the example embodiment shown in FIG. 5(c), hydrophilicity is imparted on the inner surface of distal dome 745, the inner surface of the full length of the sheath 746, and the inner surface of the full length of the inner conduit 753. Such an example embodiment frees the entire length of the catheter, from distal to proximal end, of bubbles. It is noted that the surfaces can be made hydrophilic by several methods, such as applying a hydrophilic layer or by impregnating component material with hydrophilic additives. It is further noted that component materials may be common or different for different areas within the catheter. If the component materials are different for different areas, the same or different hydrophilic layers and additives can be used, as deemed appropriate. As shown in the figure, air bubbles 702 and 751 are now free floating and will be more easily flushed out of the catheter without interfering with operation. An additional advantage to making the full length of the sheath and inner conduit hydrophilic is that the likelihood of bubble formation and adhesion along these surfaces is very low. As a result, if a bubble was to form in the proximal region, it will favorably get adhered to the proximal regions, rather than migrating into the shaft towards the distal end. If the bubble is to migrate due to the flow of the liquid, the likelihood the bubble will adhere to the inner surface of the hydrophilic shaft is very low. It is evident that air bubble 752 is still stuck on the unmodified inner surface of the proximal flush port 742.

In one example embodiment shown in FIG. 5(d), hydrophilicity is imparted on the inner surface of distal dome 745, the inner surface of the full length of the sheath 746, and the inner surface of the full length of the inner conduit 753. In addition, a hydrophilic layer 781 is applied to the torque cable 733, which lies in the outer lumen of the catheter 775. It is appropriate to use a hydrophilic layer for the torque cable in this embodiment as the torque cable is usually a metal material and cannot be impregnated with additives. Since the torque cable lies within the outer lumen of the catheter, the flushing fluid is in direct contact with the torque cable while it effluxes toward the proximal connector. The induced hydrophilicity of the torque cable will help facilitate the removal of hydrophobic air bubbles from the catheter.

FIG. 5(e) illustrates an example embodiment where surfaces are configured to be hydrophilic including the inner surface of distal dome 745, full length of the sheath 746, full length of the inner conduit 753, and inner lumen of the proximal flush port 747 of the proximal connector 741. A hydrophilic layer 781 is applied on the torque cable 733. It is noted that, the surface can be made hydrophilic by applying a hydrophilic layer or by impregnating component material with hydrophilic additives. If component materials are different for different areas, the same or different hydrophilic layers and additives may be used, as deemed appropriate. The present example embodiment is to facilitate the removal of air bubbles along the full length of the fluid path, and also at the point of insertion. When the flushing fluid is introduced, it is possible that air bubbles present in the syringe enter the catheter via the proximal flush port 742. If this was to occur, the hydrophobic bubbles would repel all surfaces configured to be hydrophilic and will exit the catheter as soon as possible. The bubble free fluid path shown in FIG. 5(d) will facilitate effective device operation and performance.

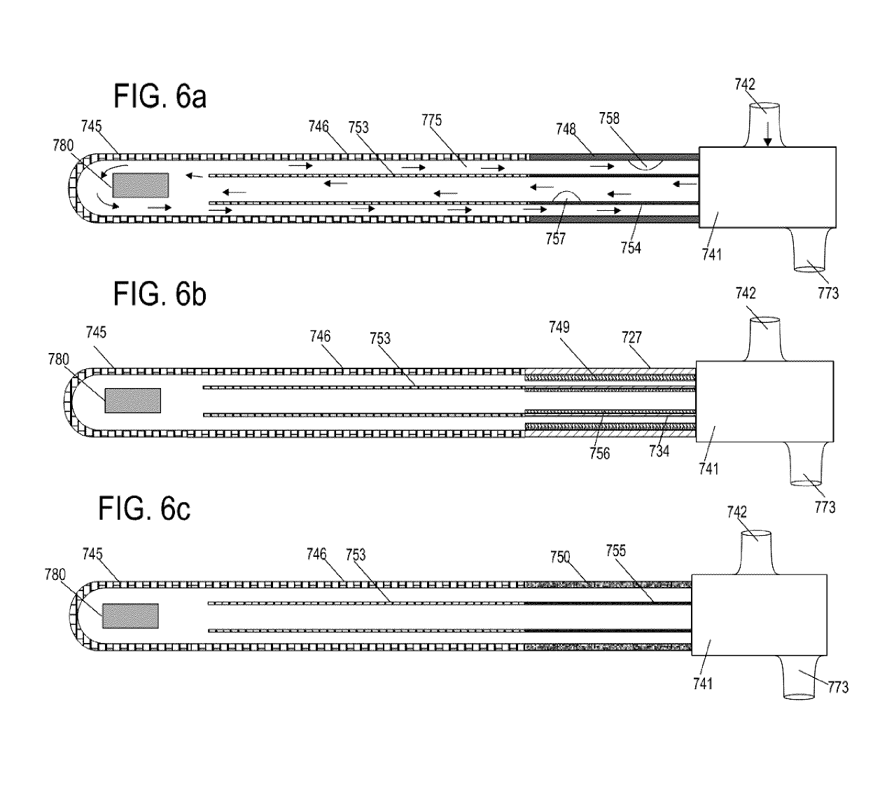

In another example embodiment, the medical probe is configured in a way to further facilitate the removal of air bubbles from a distal region, as shown in FIG. 6(a), as the distal region is the area most critical to device performance, where a functional device 780 may exist. In the present example embodiment, internal hydrophilic surfaces include the inner surfaces of the distal dome 745, a partial length of the sheath 746, and partial length of the inner conduit 753, with the remaining proximal sheath length 748 and the remaining proximal inner conduit length 754 are hydrophobic. As an example, three-quarters of the sheath and inner conduit lengths including the distal region can be configured to be hydrophilic and one-quarter of the sheath and inner conduit lengths including the proximal region can be configured to be hydrophobic.

The purpose of the hydrophilic areas is to avoid air bubble adhesion, as described above. The objective of the hydrophobic area near the proximal region of the probe is to act as a trapping area for hydrophobic air bubbles. During the point of insertion, the flushing fluid will traverse the inner lumen hydrophobic area due to pressure and convective flow. During this time, if any air bubbles exist, they will favorably adhere to the hydrophobic surface of the inner conduit 754, never migrating to other parts of the catheter. If air bubbles do happen to migrate, they will continue to move and bounce off the configured hydrophilic surfaces of the catheter. When the fluid is en route to exit the catheter through the outer lumen of the catheter 775, the hydrophobic region of sheath at proximal region 748 will further encourage bubbles to move towards this area and away from the distal end. As a result the hydrophobic proximal region of the catheter will act as trapping zone for air bubbles.

FIG. 6(a) shows a single hydrophobic bubble trapping region within each lumen. In another example implementation, one or more hydrophobic bubble trapping regions are provided in a single lumen (e.g. in the inner lumen or the outer lumen). In another example implementation, two or more hydrophobic bubble trapping regions are provided in a both lumens (e.g. in the inner lumen or the outer lumen). To fabricate such an imaging probe, the hydrophilic areas could be obtained, for example, using the methods described above, and the hydrophobic regions could be created, for example, after extrusion of materials, prior to the assembly of the final probe. Example of methods for forming hydrophobic regions on a surface are described below.

FIG. 6(b) illustrates an example embodiment where a hydrophobic bubble trapping surface is provided by applying a hydrophobic layer 749 to sheath component material and hydrophobic layer to inner conduit component material, at the proximal region 756. According to several non-limiting examples, the hydrophobic surfaces can be formed by coating the internal surfaces with any one or more of: Teflon (polytetrafluoroethylene), zinc oxide polystyrene nanocomposites, precipitated calcium carbonate, or silicone. The aforementioned processes used to apply hydrophilic coatings can also be used for hydrophobic coatings, but with different temperatures, dwell times, and curing times, as required for each specific hydrophobic material.

One example is of a hydrophobic layer is an ultra-thin siloxane-based or fluorocarbon film layer that may be formed using plasma deposited techniques. Since hydrophobic layer 749 is laterally far away from areas of energy propagation, it does not need to be acoustically or optically transparent.

In other example implementations, hydrophobic layers may be formed on limited areas of a component using the mechanical abrasion techniques described above, or, for example, the aforementioned masking techniques. As noted above, one example masking method involves the use of medical grade tape to mask a substrate from hydrophobic coating processes. Alternatively, silica desiccant (for example, consisting of an average pore size of 2.4 nanometers) can be deposited on areas to be masked from hydrophobic layers. These beads have a strong affinity for water molecules and thus will prevent any adhesion of hydrophobic layers. Once the coating process is complete, silica beads can be washed away using a concentrated alkali solvent or tetrahydrofuran.

FIG. 6(c) illustrates an example embodiment where the hydrophobicity of the surface of a component is increased by increasing the surface roughness of the component material. Based on the wettability principles of the Cassie-Baxter and Wenzel models, surface roughness makes a hydrophobic surface even more hydrophobic, and makes a hydrophilic surface even more hydrophilic. In the example embodiment shown in FIG. 6(c), the proximal portions of the sheath and inner conduit both exhibit inherent hydrophobic properties. As such the surface roughness of the sheath at the proximal region 750 and the surface roughness of the inner conduit at the proximal region 755 are increased. Increasing the surface roughness of a material implies increasing the vertical deviations of a surface from its ideal form. These deviations cause a surface to exhibit more hydrophobic properties as it increases the contact angle of aqueous solutions which come in contact with it.

Without intending to be limited by theory, it is believed that, according to the Cassie-Baxter model, an aqueous droplet sits on a surface above the rough surface features of a hydrophobic surface, where air pockets exist in the areas laterally separating the rough surface features. Thus the proximal regions of the sheath and inner conduit may be formed from a material initially having a hydrophobic surface, and where the hydrophobicity is increased by increasing the surface roughness. This increased surface roughness and associated increased hydrophobicity in the proximal region forms an air bubble trap near the proximal region to catch any bubbles which are introduced, moving in a proximal to distal direction, and also attract any bubbles which are moving in a distal to proximal direction.

It will be understood that increased surface roughness can be achieved according to a number of methods, including, but not limited to, sanding the surfaces of the polymer material after it has been extruded, incomplete drying methods performed during extrusion processes, and selecting component material which has imperfections in the mixture prior to extrusion.

As noted above, it is believed that an increase in surface roughness also increases the hydrophilicity of a hydrophilic surface. Without intending to be limited by theory, it is believed that, according to the Wenzel model, an aqueous droplet seeps into the spaces between the rough surface features of a hydrophilic surface, making the surface even more hydrophilic. Thus in one example embodiment, after a surface is made to be hydrophilic via the addition of hydrophilic additives, the surface roughness can be increased using the techniques described above.

Although FIGS. 6(a)-(c) illustrate example implementations in which approximately three-quarters of the sheath and inner conduit lengths including the distal region are configured to be hydrophilic and one-quarter of the sheath and inner conduit lengths including the proximal region can be configured to be hydrophobic, it will be understood that this embodiment is merely illustrative of a wide range of different configurations. For example, in other embodiments, the portion of the medical probe (including the distal end) that is configured to have a hydrophilic surface may be greater than or equal to approximately 1%, 2%, 5%, 10%, 20%, 30%, 40%, 50%, 60%, 70%, 80%, 90% or 95% of the total length of the probe. It is also to be understood that the embodiments shown in FIGS. 6(a) to (c) may be implemented with hydrophilic surface regions on both the inner conduit 753 and sheath 746, or one of inner conduit 753 and sheath 746.

Furthermore, the portion of one or both of the inner surface of sheath 746 or of inner conduit 753 that is hydrophilic may be split among multiple longitudinal segments (e.g. where a given segment with a hydrophilic surface is located longitudinally adjacent to neighbouring segment having a hydrophobic surface).

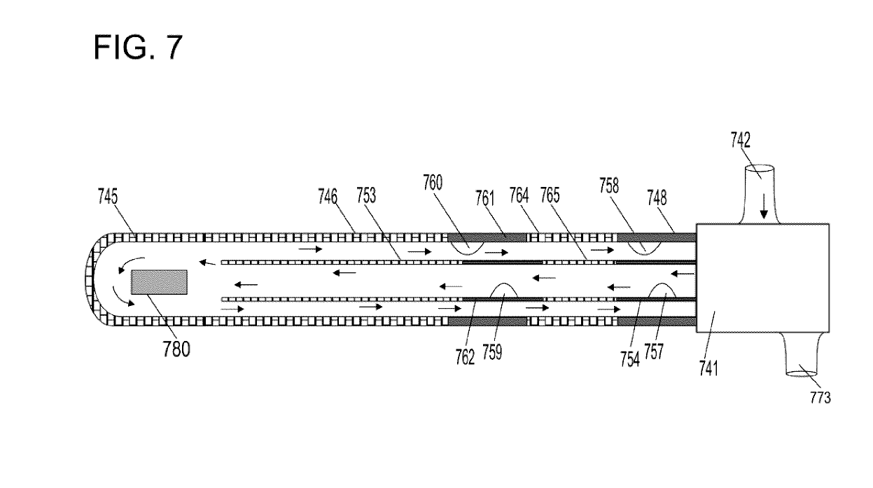

For example, in the example embodiment shown in FIG. 7, the medical probe is configured to improve the likelihood of trapping bubbles and facilitating their movement away from the functional device. The distal portion of the probe (e.g. a distal dome), and two regions of at least one of the inner surfaces of the sheath and the inner conduit, may be configured to be hydrophilic, and, two neighbouring regions of at least one of the inner surfaces of the sheath and inner conduit, away from the distal end, are configured to be hydrophobic. In the example embodiment shown in FIG. 7, the hydrophilic surfaces of the distal dome 745, sheath region one 746, inner conduit region one 753, sheath region two 764, inner conduit region two 765 are all free of bubble adhesion. These surfaces may be rendered to be hydrophilic, for example, according to any of the preceding embodiments, such as using layers or additives. If component materials are different for different portions of the medical probe, then the same or different hydrophilic layers and additives may be used.

The hydrophobic surfaces of sheath region one 748, inner conduit region one 754, sheath region two 761, inner conduit region two 762 have bubbles strongly bonded to their surfaces, shown in the FIG. 7 as bubbles 758, 757, 760, and 759, respectively. These surfaces may be configured to be hydrophobic, for example, using any of the aforementioned methods, such as hydrophobic coating layers and increasing surface roughness.

The objective of configuring alternating regions of a medical probe configured to be hydrophilic and hydrophobic is the creation of several air bubble trapping sites. In the presence of more than one bubble trapping site, the likelihood of catching bubbles increases. If bubbles traverse through a hydrophobic region during the first pass, it will likely be caught in the next hydrophobic region. As in an aforementioned embodiment, trapping sites may be provided on one or both of the inner conduit (through which the flushing fluid first traverses in the example shown) and the catheter sheath (in which the fluid backfills to exit the catheter in the example shown). In the present example embodiment, two regions are made hydrophilic and two other regions are made hydrophobic, but the alternating hydrophilic and hydrophobic regions can be extended to as large of an area as deemed appropriate. Furthermore, it will be understood that the length and locations of the hydrophobic regions on the sheath and on the inner conduit may or may not be equal or in vertical alignment. In another example implementation, several separate and pre-assembled portions of the medical probe can be configured to be hydrophilic and hydrophobic, and then the portions can be connected, bonded, attached, or otherwise coupled together to create the final assembly. In another example implementation, masking and/or mechanical abrasion techniques can be used, as described above.

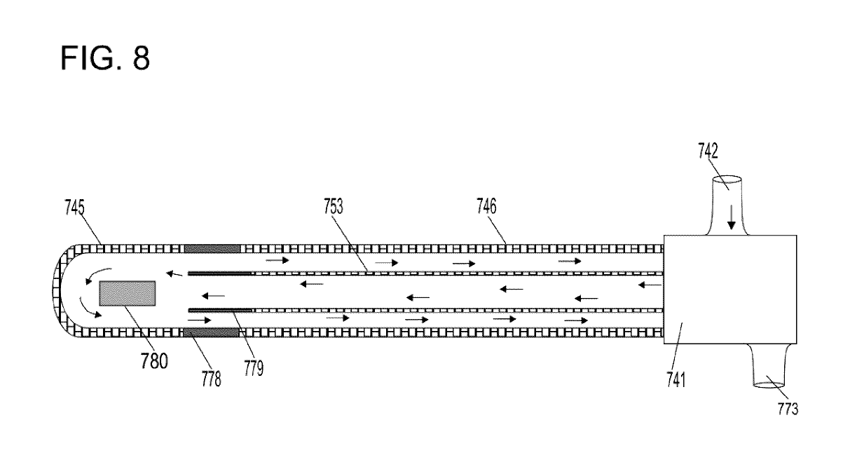

In the example embodiment shown in FIG. 8, a partial length of the sheath 778, near the location at which the 780 functional device resides during operation (e.g. near the distal end), is configured to be hydrophobic and a partial length of the inner conduit 779, near the location at which the 780 functional device resides during operation, is configured to be hydrophobic. Other surfaces are also configured to be hydrophilic, including the remaining partial length of the sheath 746, the remaining partial length of the inner conduit 753, and the inner surface of the dome 745. The purpose of such a configuration is to create a hydrophobic trapping region near the location at which the 780 functional device resides during operation, but not on the surfaces where the propagation of energy emitted by the functional device 780 occurs. This hydrophobic trapping region acts as a local trap to capture any bubbles that may reside near the distal region. These bubbles may have formed from outgassing due to turbulence, for example. Hydrophilic and hydrophobic areas can be created, for example, using any of the techniques discussed.

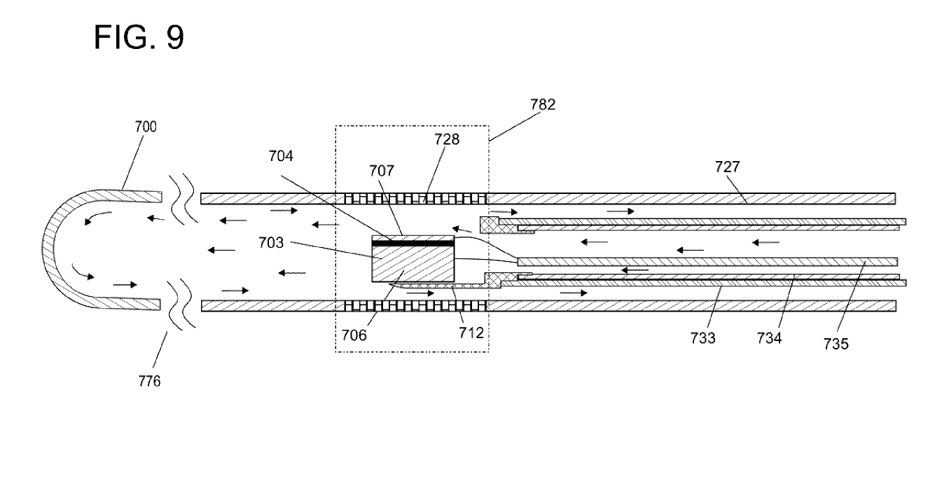

In the example embodiment shown in FIG. 9, an inner surface of an ultrasound imaging probe's sheath region 728, along its length where the transmission of ultrasound energy occurs is configured to be hydrophilic. This region may be referred to as an imaging region 782, since it is the region through which imaging energy passes. An imaging assembly, in this case an ultrasonic transducer 703, is positioned in longitudinal alignment with the imaging region 782 of the hydrophilic sheath region 728. The imaging region may be located along the sheath away from the distal dome 700 of the probe, as shown in the figure. Feature 776 shown in the drawing is a discontinuity showing that the lateral lengths of the dome and sheath can be variable, e.g. longer, but the two components may still be bonded together as described above. This surface can be configured to be hydrophilic, for example, according to any of the aforementioned methods, such as applying a hydrophilic coating layer or impregnating component material with hydrophilic additives. In this example embodiment, a region that is not immediately adjacent to the distal end is configured to be hydrophilic and the location of its application is in no way limited. In one example implementation, the imaging transducer may be longitudinally translated relative to the sheath over a pre-selected longitudinal distance during an imaging procedure while laterally imaging (not necessarily in a perpendicular direction) through the sheath, such as during a pullback procedure. In such a case, the imaging region of the sheath which is treated to be hydrophilic is also longitudinally translated and may have a length that is greater than or equal to the pre-selected longitudinal distance.

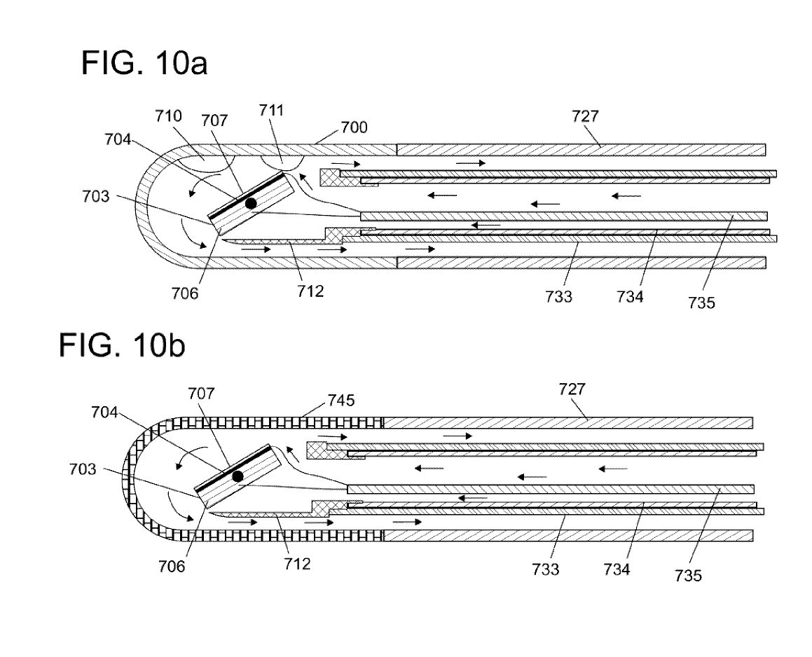

FIG. 10(a) shows an illustration of an imaging catheter in which there is a lack of internal hydrophilic surfaces, and where the ultrasonic transducer 703 is shown operating in tilted configuration at a specific angle from its original orientation. The emitting surface 707 of the transducer is tilted and aligned with position of air bubble, 710. This situation is acoustically unfavorable by impeding the passageway of acoustic waves and can thus lead to image distortion and misinterpretation. There is also an air bubble, 711 which is in the vicinity of the transducer motion and may impede the mechanical components from moving in a desired manner. Bubble migration is also possible and may lead to interference with remote components of the device. Such interference of bubbles is also possible in optical imaging probes.

FIG. 10(b) depicts an example embodiment in which internal surfaces of the imaging probe of FIG. 10(a) are configured to be hydrophilic in order to avoid the adhesion of bubbles. In particular, the inner surface of the distal dome 745 is hydrophilic resulting in the absence of air bubbles in this region. Such a configuration ensures that bubbles which specifically impede the ultrasound transducer, functionally and mechanically, are urged away from this area.

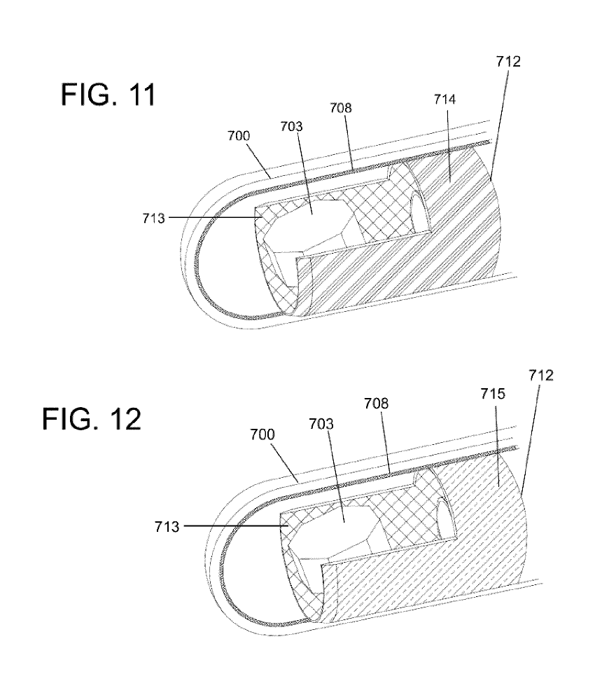

FIG. 11 shows an example imaging assembly housing 712 which houses ultrasonic transducer 703. Examples of materials which can be used to construct the imaging assembly include: various polymers such as polyether ether ketone (PEEK), Delrin®, liquid crystal polymers, Ultem™ Resin, and Xarec®. Various grades of stainless steel, metals such as gold and aluminum, and ceramics are other materials from which the imaging assembly can be built. Materials such as these are inherently hydrophobic in nature. Experiments were conducted show that air bubbles tend to stick inside, on, and around the imaging assembly housing 712. This adhesion could be as a result of the surface roughness features introduced during machining, due to the complexity of the part. Furthermore the imaging assembly housing may possess small crevices where flow of flushing fluid could be constricted, preventing convective flow from pushing air bubbles away from this area. As a result, a hydrophilic coating can be used to urge bubbles away from the surfaces of the shell, as described in the preceding example embodiments.

For example, hydrophilicity may be achieved by applying an inner hydrophilic coating 713 on an inner surface imaging assembly housing 712 and an outer hydrophilic coating 714 on an outer surface of imaging assembly housing 712 as shown in FIG. 11. In such a case, it may be appropriate to employ a hydrophilic layer to achieve the hydrophilic surface, instead of employing an embodiment in which hydrophilic additives are added to a polymer material of the imaging assembly housing, such as to not interfere with the complex fabrication and molding processes of the housing. In embodiments in which a hydrophilic coating is employed, the coating may be beneficial in enhancing the lubricity of a surface which may lower the friction of mechanical components of the shell, such as the inner surface of the shell and the mechanical mechanism holding and tilting the transducer 703. Hydrophilic coating may also be applied to the inner surface of the distal dome 708 (as described above) to further facilitate the urging of bubbles away from this area. As a result the added lubricity may also lower the friction between the distal dome and imaging assembly housing, if they were to come in contact.

FIG. 12 represents an example embodiment in which inner hydrophilic coating 713 is applied to the inner surface of imaging assembly housing 712 and to the inner surface of the distal dome 708, and an outer hydrophobic coating 715 on the outer surface of imaging assembly housing 712. According to such an example embodiment, at least a portion of the outer surface of imaging assembly housing 712 can be made hydrophobic to reduce abrasive wear on the coated inner surface of distal dome 708 that might result from anticipated variances of the shell from its desired coaxial position and orientation in the distal dome of the sheath. In such an implementation, the outer hydrophobic coating 715 on the imaging assembly 712 would prevent sticking and attraction to the hydrophilic coating on the dome 708. Additionally, the hydrophobic coating may act as a trap for air bubbles in the area. If air bubbles are free floating, they will quickly move towards the hydrophobic surface on the outer surface the imaging assembly housing and adhere favorably to it. Such movement would help with moving air bubbles away from any area where energy propagation occurs. In this case, it is most appropriate to use hydrophilic and hydrophobic layers instead of other techniques, due to the anticipated unique properties of the component material.

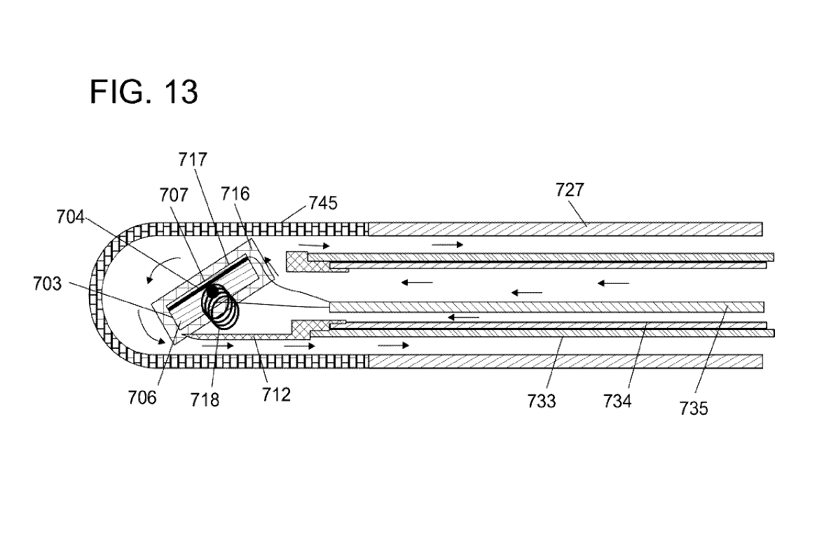

In the example embodiment shown in FIG. 13, surfaces of additional components that may reside within the catheter sheath may be rendered hydrophilic, such as by applying a hydrophilic coating. For example, at least an emitting surface of ultrasonic transducer 703 may be coated with a hydrophilic coating 716. This coating can be beneficial in removing or reducing the presence of bubbles on or near the surfaces of ultrasonic transducer 703, such as the primary transducer emitting surface 717 of ultrasonic transducer 703, which can otherwise impede image quality and overall system performance. Hydrophilic coating layer 716 may also be configured to act to reduce acoustic impedance mismatch, as described above, with hydrophilic Parylene being one such example. As described above, in one example embodiment, the hydrophilic coating layer 716 may be selected to have an acoustic impedance and a thickness to perform as an acoustic matching layer. This may avoid the need for another transducer matching layer. In one example implementation, chemical surface treatments, such a as liquid phase treatments, can be used to treat the top layer, backing layer, or both of the transducer to generate one or more hydrophilic surfaces.

Another example component where bubbles may be undesirably exist is at a mechanical spring 718 where the bubble surface tension may interfere with the proper mechanical behavior of one or more such springs used in the scanning mechanism of the probe. To resolve this, a hydrophilic coating may be applied to the spring 718 to urge bubbles away from the vicinity during operation. In this case, it may be most appropriate to use a hydrophilic layer instead of additives as the spring component material is a metal such as gold which is not readily impregnated with polymeric hydrophilic additives. The hydrophilic layer may optionally be selected to be electrically insulating which can be highly advantageous when the flushing media is conductive. One example of such a configuration would be the use of hydrophilic Parylene as the coating, with the flushing fluid being saline.

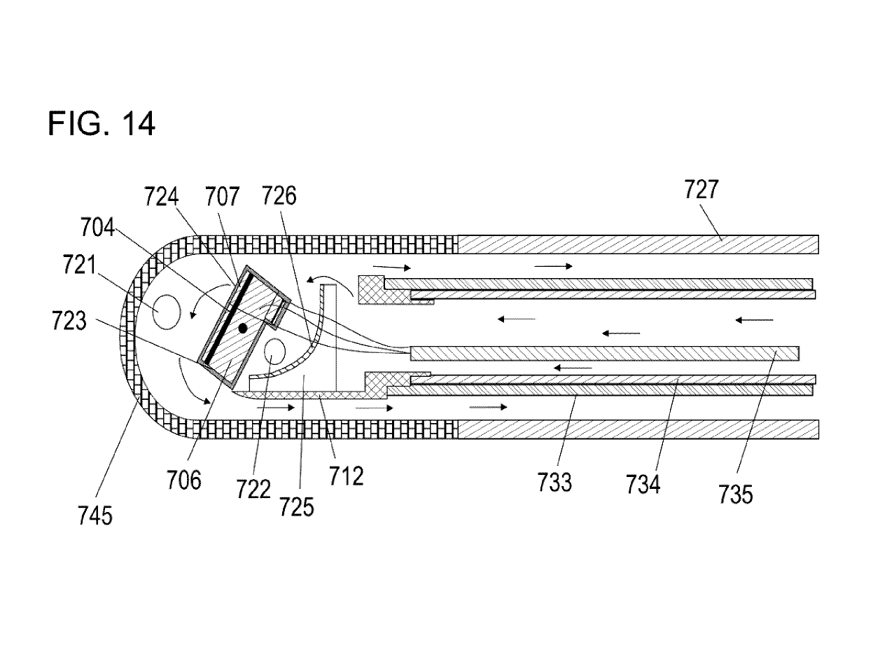

FIG. 14 represents an example embodiment in which an ultrasonic transducer within an imaging probe also includes a monolithically integrated angle detection transducer, for example, as described in PCT Patent Application No. PCT/CA2012/050057, which is incorporated herein by reference in its entirety. In the present example embodiment, the imaging transducer and the angle detection transducer (shown together at 723) are coated with a hydrophilic layer 724, the curved reflector 725 for angle detection transducer is coated with a hydrophilic layer 726, and the inner surface of the distal dome is also configured to be hydrophilic 745. For the reflector 725, the use of a hydrophilic layer may be preferred, as many materials, such as metals, may not be suitable for impregnation with additives. With regard to the formation of a hydrophilic layer on the inner surface of distal dome 745, the inner surface can be made hydrophilic, for example, by any of the aforementioned methods, a hydrophilic layer or additives may be used to make the surface hydrophilic. The hydrophilic surfaces of the transducer 724, reflector coating 726, and dome 745 promote the repulsion of air bubbles from all three surfaces. This leaves air bubble, 721, and air bubble, 722, free-floating, repelling hydrophilic surfaces, which can be forced away from the imaging area during the flush cycle. If free-floating bubble 722 were to adhere to either the curved reflector 725 or conductive backing layer 706, it may impede the tilting capability of the imaging transducer and the angle detection transducer, shown together at 723. Since the transducer 723 in the present embodiment is an oscillating component, it further creates convection flow and pushes the air bubble away. The transducer coating 724 is selected such that it does not impede the functionalities of the transducer nor the curved reflector 725. In particular, the coating is selected such that it does not spatially interfere with the tilting of the transducer and mechanism of the springs, nor does it impede the acoustic and fluid paths. This example embodiment thus involves the application of hydrophilic coatings on internal surfaces associated with both stationary and rotating components.

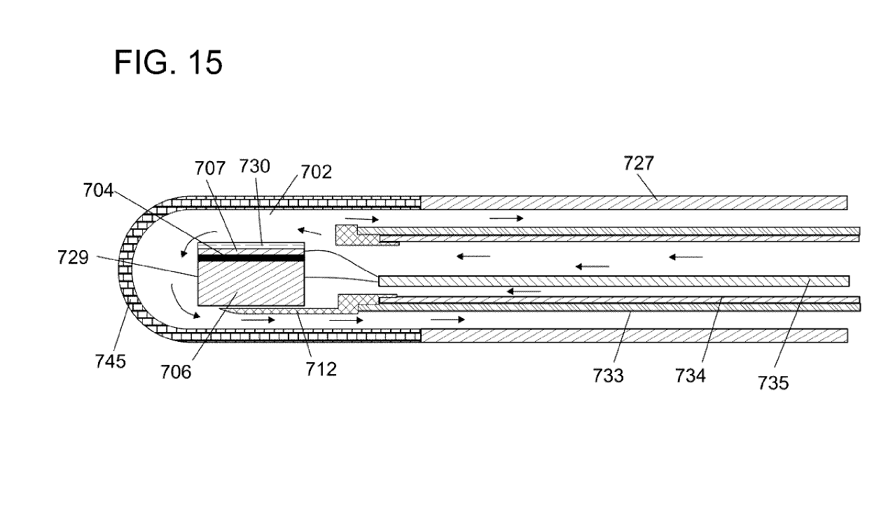

The example embodiment depicted in FIG. 15 illustrates a side-viewing ultrasound imaging transducer 729 which is selectively coated with hydrophilic coating 730 such that air bubbles are repelled from the energy emitting surface 707. A hydrophilic coating may be preferred in the present embodiment, as the transducer component material may not be alterable with hydrophilic additives. In another example implementation, chemical surface treatments may alternatively be employed to form a hydrophilic surface on the ultrasonic imaging transducer 729. Furthermore, in embodiments in which a hydrophilic coating is applied, the coating may be selected such that it does not impede, but rather may enhance the acoustic properties of the imaging modality as previously described (e.g. by reducing the acoustic impedance mismatch). As shown in the figure, the inner surface of the distal dome is also made hydrophilic 745 as described in the aforementioned embodiments.

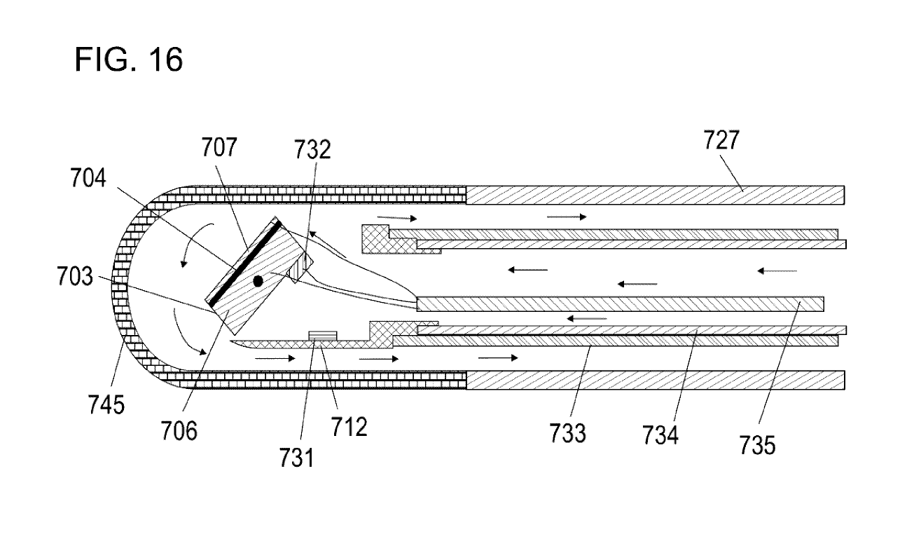

In the example embodiment shown in FIG. 16, the hydrophilic surface on an inner surface of distal dome of catheter 745 is effective in the application of magnetically driven imaging ultrasonic transducer 703, where a ferromagnetic component 731 and electromagnet 732 controls the motion of the ultrasonic transducer 703. This embodiment demonstrates that the application of hydrophilic surfaces, either rendered by coatings or additives, is not limited to imaging probes in which scanning is controlled exclusively by longitudinal rotation of the imaging probe.

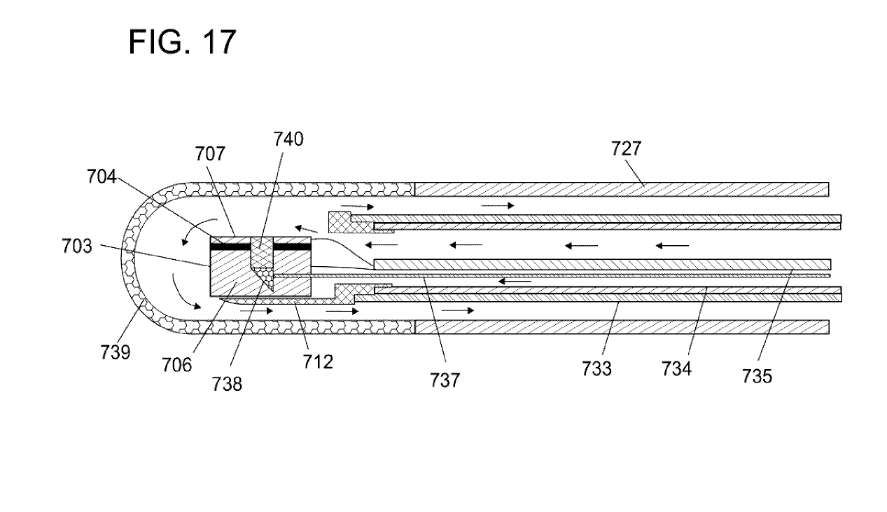

US Patent Publication No. 2008/0177183 (Courtney et al.), incorporated herein by reference in its entirety, describes embodiments for combined ultrasound and optical imaging probes. FIG. 17 represents an example embodiment in which a hydrophilic surface 739 is imparted onto the inner surface of distal dome surface of such a probe, which includes an imaging assembly capable of both acoustic and optical imaging modalities. The hydrophilic surface can be achieved, for example, via the use of hydrophilic layers or the inclusion of additives in component materials. In the example embodiment illustrated in the figure, fiber optic 737 carries optical imaging energy which is reflected by optical reflector or deflector 738 into optical guide 740, which may optionally incorporate a lens. This optical energy propagates into the catheter and can be used for imaging the catheter environs. An example optical imaging system for which this embodiment can be employed or adapted is an optical coherence tomography (OCT) system. The hydrophilic surface 739 can be formed such that it does not impede but rather enhances functionality by possessing acoustically desirable properties, as aforementioned, and is also optically transparent. Optical transparency can be characterized by measuring the optical density or percent transmission (optical power out/optical power in) of the hydrophilic material at the desired wavelength of operation. Suitable materials coating materials for providing both optical transparency and a reduction in acoustic impedance mismatch include hydrophilic Parylene, silicon dioxide based coatings, and polypropylene.

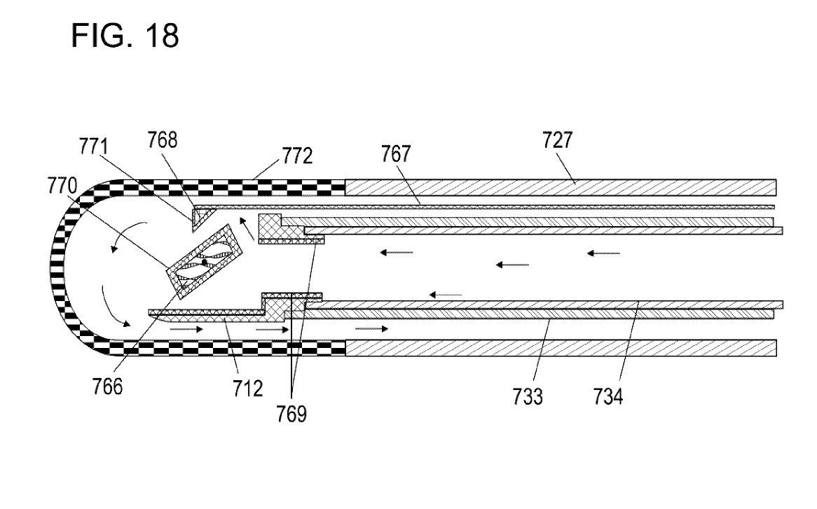

In the example embodiment shown in FIG. 18, an optical imaging probe is shown as being configured such that air bubbles are urged away from the optical imaging region, which is formed by transparent dome 772. The internal surface of transparent dome 772 is rendered hydrophilic and optically transparent. The internal surface of transparent dome 772 may be rendered hydrophilic by any suitable method that preserves the transparency of dome 772, such as adding a transparent hydrophilic layer or adding additives while forming the component such that the additives preserve the transparency of the dome after it is formed. To further facilitate the repulsion of bubbles, hydrophilic layer 769 may be applied to imaging housing assembly 712, a hydrophilic layer 770 may be applied to optical reflector 766, and/or a hydrophilic layer 771 may be applied to optical beam deflector 768 (e.g. a prism or mirror). The configuration of an optical imaging catheter as depicted in this embodiment may increase the likelihood that air bubbles do not interfere with the functionality or mechanical movement of the optical imaging modality.

Although many of the embodiments of the disclosure have been illustrated within the context of an imaging probe with a closed flushing fluidic path whereby the flush liquid is returned to the proximal region of the probe, the disclosure is not intended to be limited to such example implementations. For example, in other implementations, the medical probe may have a distal flush port, and need not include an inner lumen.

It will be understood that a medical probe according to different example embodiments of the present disclosure may include a single fluidic path extending longitudinally within the hollow sheath, or two or more fluidic paths extending in a longitudinal direction within the hollow sheath. A single fluidic path may be provided, for example, within an inner conduit, where outer lumen is not configured for liquid flow, or within a region bounded by the inner surface of the hollow sheath. In the case of a single path, the distal portion of the medical probe may include a distal port. In cases in which two or more fluidic paths are provided, the paths may be defined by two or more adjacent inner conduits, or via two or more coaxial inner conduits. Embodiments of the present disclosure can also be employed in or adapted to other types of medical probes that employ an internal fluid (such as a flushing fluid), which have a possibility of air bubbles existing within the probe and possibly interfering with its mechanical and/or functional performance. For example, medical probes that are used for imaging, therapeutic, surgical, locating and/or diagnostic purposes may employ any of the embodiments described herein.

For example, one application in which the present embodiments may be employed is high frequency ultrasound therapeutic probes. Such treatment probes could have lowered functionality in the existence of air bubbles within the probe.

One or more aspects of the embodiments described herein may also be used in an optical probe which allows for the fluorescence activation of tissue.

Another example of a medical probe that utilizes a similar flushing mechanism to the example medical probes described above is an irrigated ablation catheter, used to ablate tissue through targeted transmission of radiofrequency energy.

In another example, central venous catheters that are used to deliver nutrients and/or medicine to the body also require routine flushing procedures. It would be beneficial to adapt such catheters according to the present embodiments in order to reduce the occurrence of air bubbles, such that they do not migrate into the body.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. An imaging probe comprising:
a hollow sheath;
an imaging assembly housed within said hollow sheath and connected to an imaging conduit extending longitudinally within said hollow sheath, wherein said imaging assembly is positionable remote from a proximal region of said hollow sheath, and wherein said imaging assembly is configured to emit and/or receive imaging energy;
at least one fluidic path provided within said hollow sheath, wherein said fluidic path extends longitudinally within said hollow sheath from said proximal region and is in flow communication with said imaging assembly;
at least one port associated with said hollow sheath for introducing a liquid into said fluidic path;
wherein at least one component of said imaging assembly comprises a hydrophilic surface that is in fluid communication with said fluidic path.

2. The imaging probe according to claim 1 wherein said hydrophilic surface comprises a hydrophilic layer having an acoustic impedance that lies between an acoustic impedance of the liquid and an acoustic impedance of a material on which said hydrophilic layer resides to reduce an impedance mismatch for imaging energy propagating therethrough when said fluidic path is filled with the liquid.

3. The imaging probe according to claim 2 wherein said hydrophilic layer is configured as an acoustic matching layer for approximately matching an impedance between the liquid and a material on which said hydrophilic layer resides.

4. (canceled)

5. The imaging probe according to claim 1 wherein said at least one component comprises an ultrasonic transducer.

6. The imaging probe according to claim 1 wherein said hydrophilic surface is at least partially transparent to optical imaging energy.

7. The imaging probe according to claim 1 wherein said at least one component is a housing of said imaging assembly.

8. The imaging probe according to claim 1 wherein said imaging assembly is rotatable within said hollow sheath, and wherein at least a portion of the outer surface of said imaging assembly is hydrophobic, and wherein at least one of an inner surface of said hollow sheath adjacent to said imaging assembly and an inner surface of the imaging assembly is hydrophilic.

9. The imaging probe according to claim 1 wherein said hydrophilic surface is electrically insulating.

10. An imaging probe comprising:
a hollow sheath;
an ultrasonic transducer housed within said hollow sheath;
wherein said ultrasonic transducer is positionable remote from a proximal region of said hollow sheath, and wherein said ultrasonic transducer is configured to emit and/or receive imaging energy;
at least one fluidic path provided within said hollow sheath, wherein said fluidic path extends longitudinally within said hollow sheath from said proximal region and is in flow communication with said ultrasonic transducer; and
at least one port associated with said hollow sheath for introducing a liquid into said fluidic path;
wherein an emitting surface of said ultrasonic transducer is configured to be hydrophilic.

11. A medical probe comprising:
a hollow sheath;
an ultrasonic transducer housed within said hollow sheath, wherein said ultrasonic transducer is positionable remote from a proximal region of said hollow sheath, wherein said ultrasonic transducer is configured to emit ultrasonic energy into an external region;
at least one fluidic path provided within said hollow sheath, wherein said fluidic path extends longitudinally within said hollow sheath from said proximal region and is in flow communication with said ultrasonic transducer;
at least one port associated with said hollow sheath for introducing a liquid into said fluidic path;
wherein at least one internal surface that is in flow communication with said fluidic path, and through which the ultrasonic energy propagates from said ultrasonic transducer, comprises a hydrophilic layer having an acoustic impedance that lies between an acoustic impedance of the liquid and an acoustic impedance of a material on which said hydrophilic layer resides to reduce an impedance mismatch for ultrasonic energy propagating through said internal surface when said fluidic path is filled with a liquid.

12. A medical probe comprising:
a hollow sheath;
a functional device housed within said hollow sheath, wherein said functional device is rotatable and positionable remote from a proximal region of said hollow sheath;
at least one fluidic path provided within said hollow sheath, wherein said fluidic path extends longitudinally within said hollow sheath from said proximal region and is in flow communication with said functional device;
at least one port associated with said hollow sheath for introducing a liquid into said fluidic path;
wherein one or more stationary surfaces within said hollow sheath that are in fluid communication with said fluidic path is configured to be hydrophilic over at least a portion thereof; and
wherein one or more rotatable components within said hollow sheath comprises a rotatable surface in fluid communication with said fluidic path that is configured such that at least a portion of said rotatable surface is hydrophilic.

13. A medical probe comprising:
a hollow sheath;
a functional device housed within said hollow sheath, wherein said functional device is positionable remote from a proximal region of said hollow sheath;
at least one fluidic path provided within said hollow sheath, wherein said fluidic path extends longitudinally within said hollow sheath from said proximal region and is in flow communication with said functional device;
at least one port associated with said hollow sheath, wherein said port is in flow communication with said fluidic path;
wherein at least one internal surface defining said fluidic path comprises:
at least one hydrophilic surface region for reducing adhesion of bubbles that could impair the operation of said functional device, wherein at least a portion of said at least one hydrophilic surface region is provided near said functional device when said functional device is employed during a medical procedure; and
at least one hydrophobic surface region for trapping bubbles and preventing bubbles from interfering with the operation of said functional device, wherein said at least one hydrophobic surface region is provided at a location between said functional device and said port.

14. The medical probe according to claim 13 wherein said fluidic path is defined, at least in part, by an inner surface of said hollow sheath.

15. (canceled)

16. The medical probe according to claim 14 wherein two or more hydrophilic surface regions are provided on said inner surface of said hollow sheath, where a hydrophobic surface region is provided between hydrophilic surface regions.

17. (canceled)

18. The medical probe according to claim 14 further comprising an inner fluidic conduit housed within said hollow sheath;
wherein said fluidic path is defined by an outer lumen formed between:
an inner surface of said hollow sheath and an outer surface of said inner fluidic conduit; and
an inner lumen formed within said inner fluidic conduit;
wherein said inner lumen is in fluid communication with said outer lumen near a region remote from the proximal end;
wherein said port is a first port;
wherein one of said inner lumen and said outer lumen is in fluid communication with said first port; and
wherein another of said inner lumen and said outer lumen is in fluid communication with a second port.

19-27. (canceled)

28. The medical probe according to claim 14 wherein said medical probe further comprises a torque cable housed within said hollow sheath for rotating said functional device, and wherein at least a portion of a surface of said torque cable is a hydrophilic surface.

29. The medical probe according to claim 14 wherein said functional device is an imaging device.

30. A medical probe comprising:
a hollow sheath;
a functional device housed within said hollow sheath, wherein said functional device is rotatable and positionable remote from a proximal region of said hollow sheath;
at least one fluidic path provided within said hollow sheath, wherein said fluidic path extends longitudinally within said hollow sheath from said proximal region and is in flow communication with said functional device;
at least one port associated with said hollow sheath for introducing a liquid into said fluidic path;
wherein one or more rotatable components housed within the hollow sheath that have a rotatable surface in fluid communication with said fluidic path is configured such that at least a portion of said rotatable surface is hydrophilic.

31. A medical probe comprising:
a hollow sheath;
a functional assembly housed within said hollow sheath, wherein said functional assembly is positionable remote from a proximal region of said hollow sheath, and wherein said functional assembly is configured to emit and/or receive energy;
at least one fluidic path provided within said hollow sheath, wherein said fluidic path extends longitudinally within said hollow sheath from said proximal region and is in flow communication with said functional assembly;
at least one port associated with said hollow sheath for introducing a liquid into said fluidic path;
wherein at least one surface that is in flow communication with said fluidic path comprises a hydrophilic layer having an acoustic impedance that lies between an acoustic impedance of the liquid and an acoustic impedance of a material on which said hydrophilic layer resides to reduce an impedance mismatch for energy propagating therethrough when said fluidic path is filled with a liquid.

32. The imaging probe according to claim 1 wherein said at least one component comprises a tiltable component.

33. The imaging probe according to claim 5 wherein said ultrasonic transducer comprises a tiltable imaging transducer and an angle detection transducer configured to measure the tilt angle of the tiltable imaging transducer, and wherein one of the imaging transducer and the angle detection transducer has a hydrophilic surface.

34. (canceled)

35. The imaging probe according to claim 1 any one of claims 1 to 9 wherein said at least one component of said imaging assembly comprises at least one rotatable component having a rotatable surface in fluid communication with said fluidic path, wherein at least a portion of said rotatable surface is hydrophilic.

36. (canceled)

37. The imaging probe according to claim 1 wherein said fluidic path is a closed flushing fluidic path whereby the flush liquid is returned to the proximal region of the imaging probe.

38. The imaging probe according to claim 12 wherein said fluidic path is a closed flushing fluidic path whereby the flush liquid is returned to the proximal region of the imaging probe.
